(19) United States
(12) Patent Application Publication
(10) Pub. No.: US 2025/0135007 A1
(43) Pub. Date: May 1, 2025

(54) COMPOSITIONS COMPRISING STEROL-AMINO-PHOSPHATE COMPOUNDS AND METHODS OF MAKING AND USE THEREOF

(71) Applicants: Ron WEISS, Cambridge, MA (US); Ohio State Innovation Foundation, Columbus, OH (US); MASSACHUSETTS INSTITUTE OF TECHNOLOGY, Cambridge, MA (US)

(72) Inventors: Yizhou DONG, New York, NY (US); Shi DU, New York, NY (US); Wenqing LI, Columbus, OH (US); Yuebao ZHANG, Columbus, OH (US); Darrell IRVINE, Cambridge, MA (US); Ron WEISS, Cambridge, MA (US)

(21) Appl. No.: 18/722,113

(22) PCT Filed: Dec. 20, 2022
(86) PCT No.: PCT/US2022/053484

§ 371 Date: Jun. 20, 2024

Related U.S. Application Data
(60) Provisional application No. 63/291,676, filed on Dec. 20, 2021.

(51) Int. Cl. A61K 47/28; A61K 9/00; A61K 31/7105; A61P 15/10

(52) U.S. Cl. CPC A61K 47/28; A61K 9/0019; A61K 9/0034; A61K 31/7105; A61P 15/10

(57) ABSTRACT

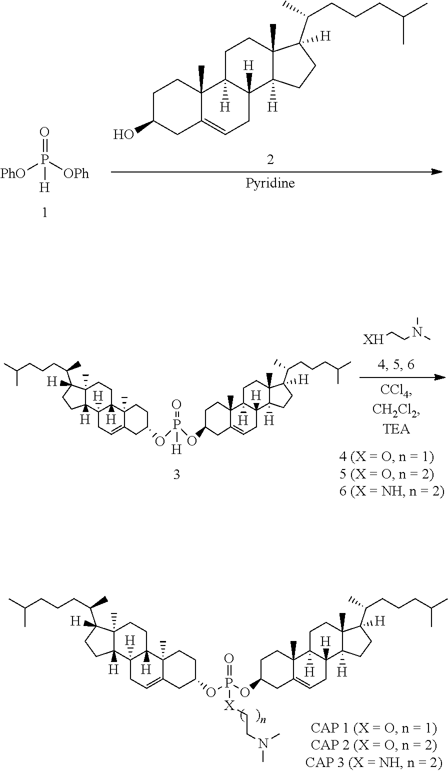

Disclosed herein are compositions comprising sterol-amino-phosphate compounds and methods of making and use thereof. Also disclosed herein are methods of making and methods of use of the compounds, compositions, and/or lipid particles disclosed herein. For example, the compounds and compositions are useful for treating a disease or disorder in humans and in animals. In some examples, the subject is a human male and the disease or disorder comprises male infertility.

### CROSS-REFERENCE TO RELATED APPLICATIONS

This application claims the benefit of priority to U.S. Provisional Application No. 63/291,676, filed Dec. 20, 2021, which is hereby incorporated herein by reference in its entirety.

### GOVERNMENT SUPPORT CLAUSE

This invention was made with government support under grant/contract number R35 GM119679 and EB025854 awarded by the National Institutes of Health. The government has certain rights in the invention.

### BACKGROUND

Male infertility caused by genetic mutations has become an important type of infertility. Currently, there is no reliable method in the clinic to address this medical need. The emergence of messenger RNA (mRNA) therapy provides a possible strategy for restoring mutant genes in the reproductive system. However, effective delivery of mRNA to spermatocytes remains a formidable challenge. The compositions and methods discussed herein address this and other needs.

### SUMMARY

In accordance with the purposes of the disclosed devices and methods as embodied and broadly described herein, the disclosed subject matter relates to compositions comprising sterol-amino-phosphate compounds and methods of making and use thereof.

For example, disclosed herein are compositions comprising a compound defined by Formula I-Formula V, or a pharmaceutically acceptable salt thereof.

Also disclosed herein are lipid particles comprising any of the compounds or compositions disclosed herein.

Also disclosed herein are pharmaceutical compositions comprising any of the compounds, compositions, and/or lipid particles disclosed herein.

Also disclosed herein are methods of making and methods of use of the compounds, compositions, and/or lipid particles disclosed herein.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic illustration of delivery of mRNA encoding Dmc1 using cholesterol-amino-phosphate (CAP) derived lipid nanoparticles (CAP LNPs). CAP LNPs are formulated with CAP, DOPE, DMG-PEG, and mRNA encoding Dmc1. After microinjection to seminiferous tubules, CAP LNPs deliver Dmc1 mRNA to spermatocytes, which recover the chromosome recombination as well as spermatogenesis.

FIG. 2A. Synthesis and structures of CAP compounds.

FIG. 2B. 3D structures of CAP2 and MC3 lipids.

FIG. 2C. Calculated parameters and P values of CAP2 and MC3 lipids.

FIG. 2D. Size and polydispersity index (PDI) of CAP2-4 LNPs measured by DLS.

FIG. 2E. Encapsulation efficiency and zeta potential of CAP2-4 LNPs.

FIG. 2F. Representative Cryo-EM image of CAP2-4 LNPs. Scale bar, 50 nm.

FIG. 2G. Confocal images of Hep3B cells incubated with calcein alone or with CAP2-4 LNPs.

FIG. 2H. Cellular uptake of CAP2-4 LNPs encapsulating Alexa-Fluor-647-labeled RNAs in the presence of endocytosis inhibitors, EIPA, MBCD and CPZ. Data are presented as the mean±S.D. (n=3). Statistical significance was analyzed by one-way ANOVA with Dunnett's multiple comparison test. * P<0.05, ** P<0.01.

FIG. 2I. Representative fluorescence microscopy images of seminiferous tubules from CAP2-4 LNPs and MC3 LNPs administrated mice in comparison to untreated mice (n=3). Scale bar: 50 μm.

FIG. 3A. Delivery of traditional mRNA or saRNA encoding Dmc1 protein in Dmc14-mice. Fluorescence microscopy of representative seminiferous tubules 24 hours after administration with CAP2-4 LNPs encapsulating FLAG-tagged Dmc1 mRNA and FLAG-tagged Dmc1 saRNA in comparison to the untreated group (n=3). Scale bar: 50 μm.

FIG. 3B. Delivery of traditional mRNA or saRNA encoding Dmc1 protein in Dmc1-mice. Fluorescence microscopy of representative seminiferous tubules 72 hours after administration with CAP2-4 LNPs encapsulating FLAG-tagged Dmc1 mRNA and FLAG-tagged Dmc1 saRNA in comparison to the untreated group (n=3). Scale bar: 50 μm.

FIG. 3C. Luciferase expression mediated by FLuc saRNA encapsulated CAP2-4 LNPs in the testes. saRNA-LNPs were microinjected into the left testis and the luciferase expression from the treated side (left testis) and untreated side (right testis) was monitored for 10 days. Data are presented as the mean #S.D. (n=3). Statistical significance was analyzed by the two-tailed Student's t-test. ** P<0.01, *** P<0.001, **** P<0.0001

FIG. 4A. Chromosome spreading assay of spermatocytes from wild-type (WT) mice. SC was categorized by staining chromosomes with anti-SYCP3 antibody (green) and SYCP1 antibody (red).

FIG. 4B. Chromosome spreading assay of spermatocytes from CAP2-4 Dmc1 saRNA LNPs treated Dmc1^{-/-}mice. SC was categorized by staining chromosomes with anti-SYCP3 antibody (green) and SYCP1 antibody (red).

FIG. 4C. Chromosome spreading assay of spermatocytes from untreated Dmc1^{-/-}mice. SC was categorized by staining chromosomes with anti-SYCP3 antibody (green) and SYCP1 antibody (red).

FIG. 4D. Percentage of spermatocytes in different substages are indicated in the pie charts and numbers of spermatocytes at each substage are indicated in a column chart (n=3). All data are presented as mean #s.d.

FIG. 5A. Fluorescent images of PNA-lectin labeled spermatozoa of WT; Dmc1^{-/-}mice treated with CAP2-4 LNPs, and untreated Dmc1^{-/-}mice. Scale bar: 50 μm.

FIG. 5B. The mean number of PNA positive cells per seminiferous tubules of WT mice, Dmc1^{-/-}mice treated with CAP2-4 LNPs and Dmc1^{-/-}mice. All data are representative images from n=3 independent samples and all the statistical analysis are presented as mean±s.d. (n=3). Statistical significance was analyzed by the two-tailed Student's t-test. *P<0.05, ** P<0.01, **** P<0.0001.

FIG. 5C. Histological analysis of WT; Dmc1^{-/-}mice treated with CAP2-4 LNPs, and untreated Dmc1^{-/-}mice. The black box indicates images at magnifications of 20x. Scale bar: 50 μm.

FIG. 5D. The average geminate layer thickness of WT; Dmc1^{-/-}mice treated with CAP2-4 LNPs, and untreated Dmc1-mice. All data are representative images from n=3 independent samples and all the statistical analysis are presented as mean±s.d. (n=3). Statistical significance was analyzed by the two-tailed Student's t-test. * P<0.05, ** P<0.01, **** P<0.0001.

FIG. 5E. The diameter of seminiferous tubes of WT; Dmc1^{-/-}mice treated with CAP2-4 LNPs, and untreated Dmc1^{-/-}mice. All data are representative images from n=3 independent samples and all the statistical analysis are presented as mean±s.d. (n=3). Statistical significance was analyzed by the two-tailed Student's t-test. * P<0.05, ** P<0.01, **** P<0.0001.

FIG. 5F. TUNEL assay of testes of WT; Dmc1^{-/-}mice treated with CAP2-4 LNPs, and untreated Dmc1^{-/-}mice. Scale bar: 50 μm.

FIG. 5G. The mean number of apoptotic cells per area of WT mice, Dmc17 mice treated with CAP2-4 LNPs and Dmc1^{-/-}mice. All data are representative images from n=3 independent samples and all the statistical analysis are presented as mean±s.d. (n=3). Statistical significance was analyzed by the two-tailed Student's t-test. * P<0.05, ** P<0.01, **** P<0.0001.

FIG. 6. Size and PDI of CAP-LNPs and MC3 LNPs measured by DLS.

FIG. 7. In vitro delivery efficiency of FLuc mRNA encapsulated CAP-LNPs as compared to MC3 LNPs in Hep3B cells.

FIG. 8. Fluorescent images of PNA-lectin labeled spermatozoa of WT; Dmc1^{-/-}mice treated with CAP2-4 LNPs, and untreated Dmc14 mice (testes). Scale bar: 50 μm.

FIG. 9. Fluorescent images of PNA-lectin labeled spermatozoa of WT; Dmc1^{-/-}mice treated with CAP2-4 LNPs, and untreated Dmc1 ^{-/-}mice (epididymis). Scale bar: 50 μm.

FIG. 10. Histological analysis of WT; Dmc1^{-/-}mice treated with CAP2-4 LNPs, and untreated Dmc1^{-/-}mice at 20× magnifications. Scale bar: 50 μm.

FIG. 11. Histological analysis of WT; Dmc1^{-/-}mice treated with CAP2-4 LNPs, and untreated Dmc1^{-/-}mice at 100× magnifications. The yellow arrows mark the mature spermatids. Scale bar: 5 μm.

FIG. 12. Representation of sperms from Dmc1^{-/-}mice treated with CAP2-4 LNPs (A-E), and microscopy analysis of untreated Dmc17 mice. Scale bar: 20 μm.

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of fibrotic conditions, “treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “anticancer” refers to the ability to treat or control cellular proliferation and/or tumor growth at any concentration.

As used herein, “molecular weight” refers to number average molecular weight as measured by ¹H NMR spectroscopy, unless indicated otherwise.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

As used herein, the term “encapsulation,” or grammatical equivalent, refers to the process of confining an individual nucleic acid molecule within a nanoparticle.

As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used interchangeably.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. “Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄)alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-bexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹²²) C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₀, C₂-C₁₄, C₂-C₁₂, C₂-C₁₀, C₂-C₈, C₂-C₆, or C₂-C₄)alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula-C(O) Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹z²) C(═OZ³) (═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, henoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula-C(O) H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

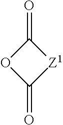

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O) O.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O) OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

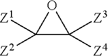

where Z¹, Z², z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O) Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO2.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, beterocycloalkyl, or heterocycloalkenyl group described above.

The term “silyl” as used herein is represented by the formula —SiZ¹z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above. The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹” “R²,” “R³” “R^{B}” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds and methods of making and use thereof. For example, disclosed herein are compositions comprising a compound defined by Formula I, or a pharmaceutically acceptable salt thereof:

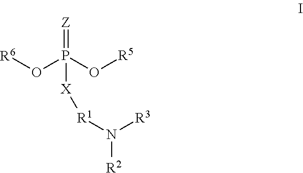

wherein
X is O, S, CH₂, or NR⁴;
Z is O or S;
R¹ is substituted or unsubstituted C₁-C₈ alkyl, substituted or unsubstituted C₁-C₅ alkenyl, or substituted or unsubstituted C₁-C₈ alkynyl;
R² and R³ are independently hydrogen or substituted or unsubstituted C₁-C₈ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety;
R⁴, when present, is hydrogen or substituted or unsubstituted C₁-C₈ alkyl; and
R⁵ and R⁶ are independently a sterol.

In some examples of Formula I, X is NR⁴. In some examples of Formula I, X is NR⁴ and R⁴ is hydrogen. In some examples of Formula I, X is NR⁴ and R⁴ is substituted or unsubstituted C₁-C₈ alkyl. In some examples of Formula I, X is NR⁴ and R⁴ is substituted C₁-C₈ alkyl, such as C₁-C₅ hydroxyalkyl. In some examples of Formula I, X is O.

In some examples of Formula I, Z is O.

In some examples of Formula I, R¹ is substituted or unsubstituted C₁-C₈ alkyl. In some examples of Formula I, R¹ is a substituted or unsubstituted C₂-C₄ alkyl. In some examples of Formula I, R¹ is an unsubstituted C₂-C₄ alkyl.

In some examples of Formula I, R² and R³ are each independently substituted or unsubstituted C₁-C₈ alkyl. In some examples of Formula I, R² and R³ are each independently substituted C₁-C₈ alkyl, such as C₁-C₅ hydroxyalkyl. In some examples of Formula I, R² and R³ are each independently substituted or unsubstituted C₁-C₈ alkyl. In some examples of Formula I, R² and R³ are each independently unsubstituted C₁-C₃ alkyl. In some examples of Formula I, R² and R³ are the same. In some examples of Formula IV, R² and R³ are both CH₃.

In some examples of Formula I, R⁵ and R⁶ are each independently selected from the group consisting of cholesterol, β-sitosterol, fucosterol, campesterol, ergosterol, stigmastanol, brassicasterol, and derivatives thereof. In some examples of Formula I, R⁵ and Re are the same. In some examples of Formula I, R⁵ and R⁶ are both cholesterol or a derivative thereof.

In some examples, the compound is defined by Formula II, or a pharmaceutically acceptable salt thereof:

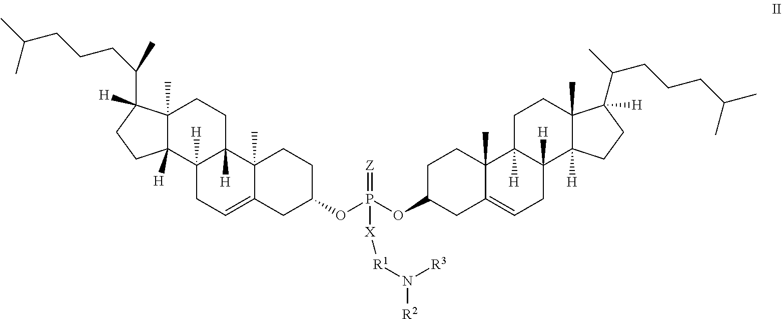

wherein
X is O, S, CH₂, or NR⁴;
Z is O or S;
R¹ is substituted or unsubstituted C₁-C₈ alkyl, substituted or unsubstituted C₁-C₅ alkenyl, or substituted or unsubstituted C₁-C₈ alkynyl;
R² and R³ are independently hydrogen or substituted or unsubstituted C₁-C₅ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety; and
R⁴, when present, is hydrogen or substituted or unsubstituted C₁-C₈ alkyl.

In some examples of Formula II, X is NR⁴. In some examples of Formula II, X is NR⁴ and R⁴ is hydrogen. In some examples of Formula II, X is NR⁴ and R⁴ is substituted or unsubstituted C₁-C₈ alkyl. In some examples of Formula II, X is NR⁴ and R⁴ is substituted C₁-C₅ alkyl, such as C₁-C₅ hydroxyalkyl. In some examples of Formula II, X is O.

In some examples of Formula II, Z is O.

In some examples of Formula II, R¹ is substituted or unsubstituted C₁-C₈ alkyl. In some examples of Formula II, R¹ is a substituted or unsubstituted C₂-C₄ alkyl. In some examples of Formula II, R¹ is an unsubstituted C₂-C₄ alkyl.

In some examples of Formula II, R² and R³ are each independently substituted or unsubstituted C₁-C₈ alkyl. In some examples of Formula II, R² and R³ are each independently substituted C₁-C₈ alkyl, such as C₁-C₅ hydroxyalkyl. In some examples of Formula II, R² and R³ are each independently substituted or unsubstituted C₁-C₃ alkyl. In some examples of Formula II, R² and R³ are each independently unsubstituted C₁-C₃ alkyl. In some examples of Formula II, R² and R³ are the same. In some examples of Formula II, R² and R³ are both CH₃.

In some examples, the compound is defined by Formula III, or a pharmaceutically acceptable salt thereof:

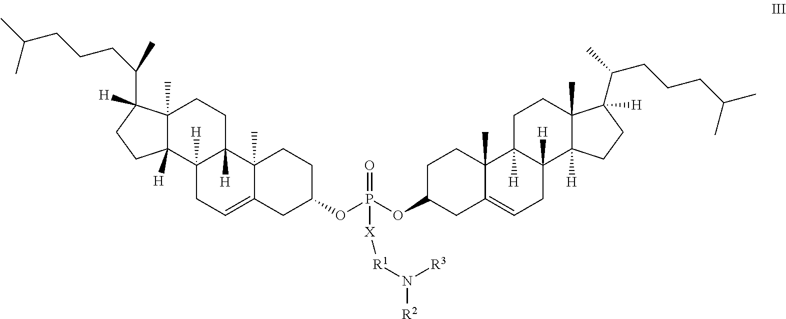

wherein
X is O, S, CH₂, or NR⁴;
R¹ is substituted or unsubstituted C₁-C₈ alkyl, substituted or unsubstituted C₁-C₅ alkenyl, or substituted or unsubstituted C₁-C₈ alkynyl;
R² and R³ are independently hydrogen or substituted or unsubstituted C₁-C₅ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety; and
R⁴, when present, is hydrogen or substituted or unsubstituted C₁-C₈ alkyl.

In some examples of Formula III, X is NR⁴. In some examples of Formula III, X is NR⁴ and R⁴ is hydrogen. In some examples of Formula III, X is NR⁴ and R⁴ is substituted or unsubstituted C₁-C₈ alkyl. In some examples of Formula III, X is NR⁴ and R⁴ is substituted C₁-C₅ alkyl, such as C₁-C₅ hydroxyalkyl. In some examples of Formula III, X is O.

In some examples of Formula III, R¹ is substituted or unsubstituted C₁-C₈ alkyl. In some examples of Formula III, R¹ is a substituted or unsubstituted C₂-C₄ alkyl. In some examples of Formula III, R¹ is an unsubstituted C₂-C₄ alkyl.

In some examples of Formula III, R² and R³ are each independently substituted or unsubstituted C₁-C₈ alkyl. In some examples of Formula III, R² and R³ are each independently substituted C₁-C₈ alkyl, such as C₁-C₅ hydroxyalkyl. In some examples of Formula III, R² and R³ are each independently substituted or unsubstituted C₁-C₃ alkyl. In some examples of Formula III, R² and R³ are each independently unsubstituted C₁-C₃ alkyl. In some examples of Formula III, R² and R³ are the same. In some examples of Formula III, R² and R³ are both CH₃.

In some examples, the compound is defined by Formula IV, or a pharmaceutically acceptable salt thereof:

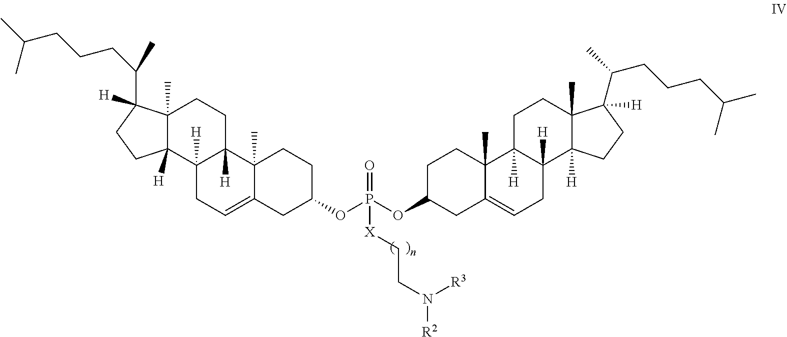

wherein
X is O, S, CH₂, or NR⁴;
R² and R³ are independently hydrogen or substituted or unsubstituted C₁-C₅ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety;
R⁴, when present, is hydrogen or substituted or unsubstituted C₁-C₈ alkyl; and
n is an integer from 1 to 5 (e.g., 1, 2, 3, 4, or 5).

In some examples of Formula IV, X is NR⁴. In some examples of Formula IV, X is NR⁴ and R⁴ is hydrogen. In some examples of Formula IV, X is NR⁴ and R⁴ is substituted or unsubstituted C₁-C₈ alkyl. In some examples of Formula IV, X is NR⁴ and R⁴ is substituted C₁-C₅ alkyl, such as C₁-C₅ hydroxyalkyl. In some examples of Formula IV, X is O).

In some examples of Formula IV, R² and R³ are each independently substituted or unsubstituted C₁-C₈ alkyl. In some examples of Formula IV, R² and R³ are each independently substituted C₁-C₈ alkyl, such as C₁-C₅ hydroxyalkyl. In some examples of Formula IV, R² and R³ are each independently substituted or unsubstituted C₁-C₃ alkyl. In some examples of Formula IV, R² and R³ are each independently unsubstituted C₁-C₃ alkyl. In some examples of Formula IV, R² and R³ are the same. In some examples of Formula IV, R² and R³ are both CH₃.

In some examples of Formula IV, n is an integer of from 1 to 3 (e.g., 1, 2, or 3). In some examples of Formula IV, n is an integer from 1 to 2 (e.g., 1 or 2). In some examples of Formula IV, n is 1. In some examples of Formula IV, n is 2.

In some examples of Formula IV, X is O and n is 1.

In some examples, the compound is defined by Formula V, or a pharmaceutically acceptable salt thereof:

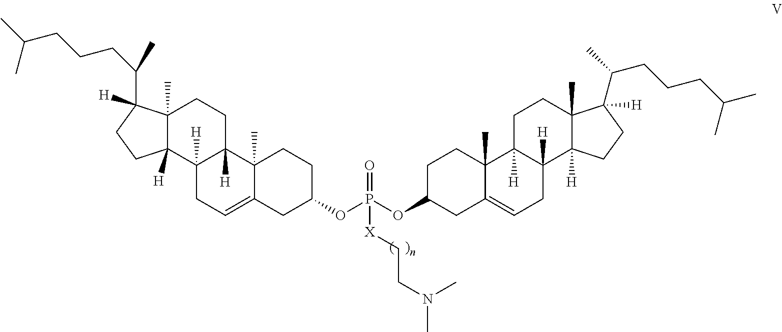

wherein
X is O, S, CH₂, or NR⁴;
R⁴, when present, is hydrogen or substituted or unsubstituted C₁-C₈ alkyl; and
n is an integer from 1 to 5 (e.g., 1, 2, 3, 4, or 5).

In some examples of Formula V, X is NR⁴. In some examples of Formula V, X is NR⁴ and R⁴ is hydrogen. In some examples of Formula V, X is NR⁴ and R⁴ is substituted or unsubstituted C₁-C₈ alkyl. In some examples of Formula V, X is NR⁴ and R⁴ is substituted C₁-C₅ alkyl, such as C₁-C₅ hydroxyalkyl. In some examples of Formula V, X is O.

In some examples of Formula V, n is an integer of from 1 to 3 (e.g., 1, 2, or 3). In some examples of Formula V, n is an integer from 1 to 2 (e.g., 1 or 2). In some examples of Formula V, n is 1. In some examples of Formula V, n is 2.

In some examples of Formula V, X is O and n is 1.

In some examples, the compound is selected from the group consisting of:

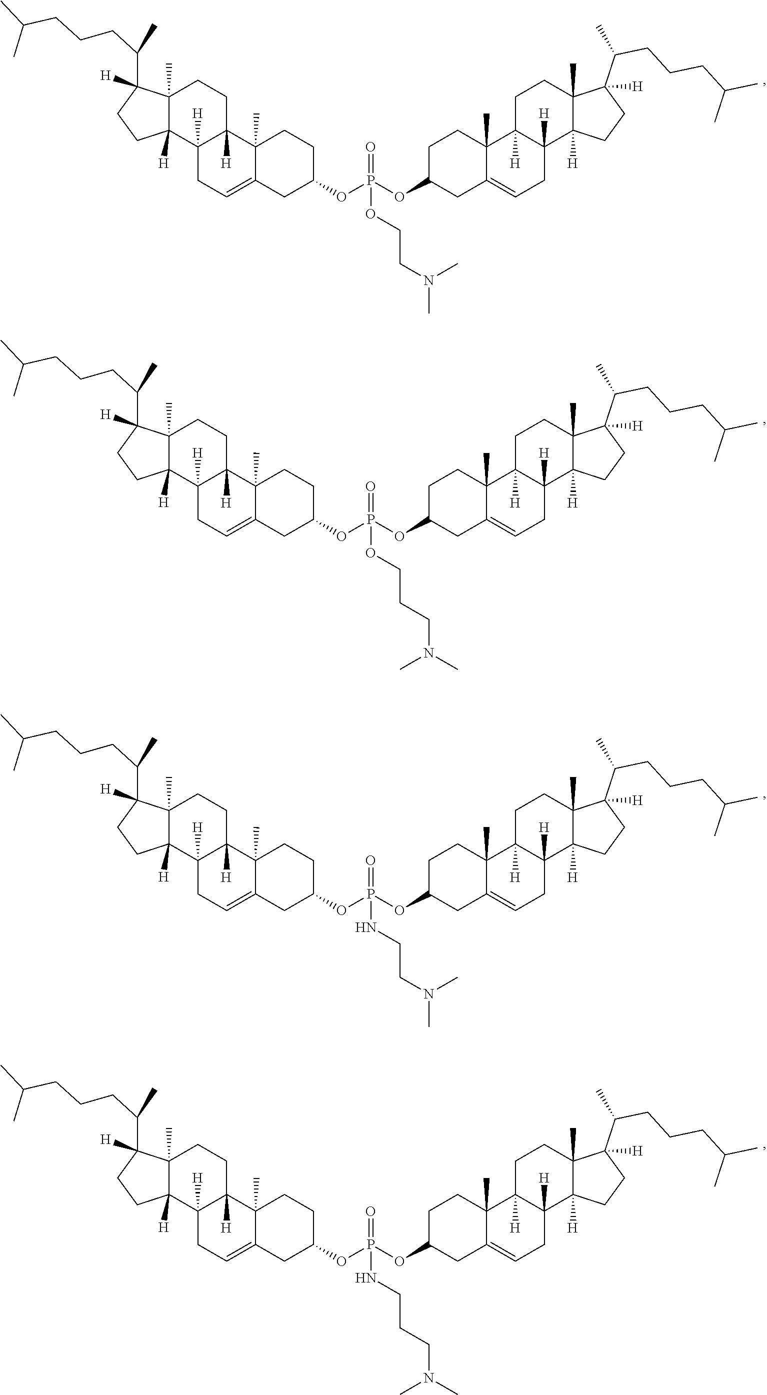

pharmaceutically acceptable salts thereof, and combinations thereof.

In some examples, the compound is selected from the group consisting of:

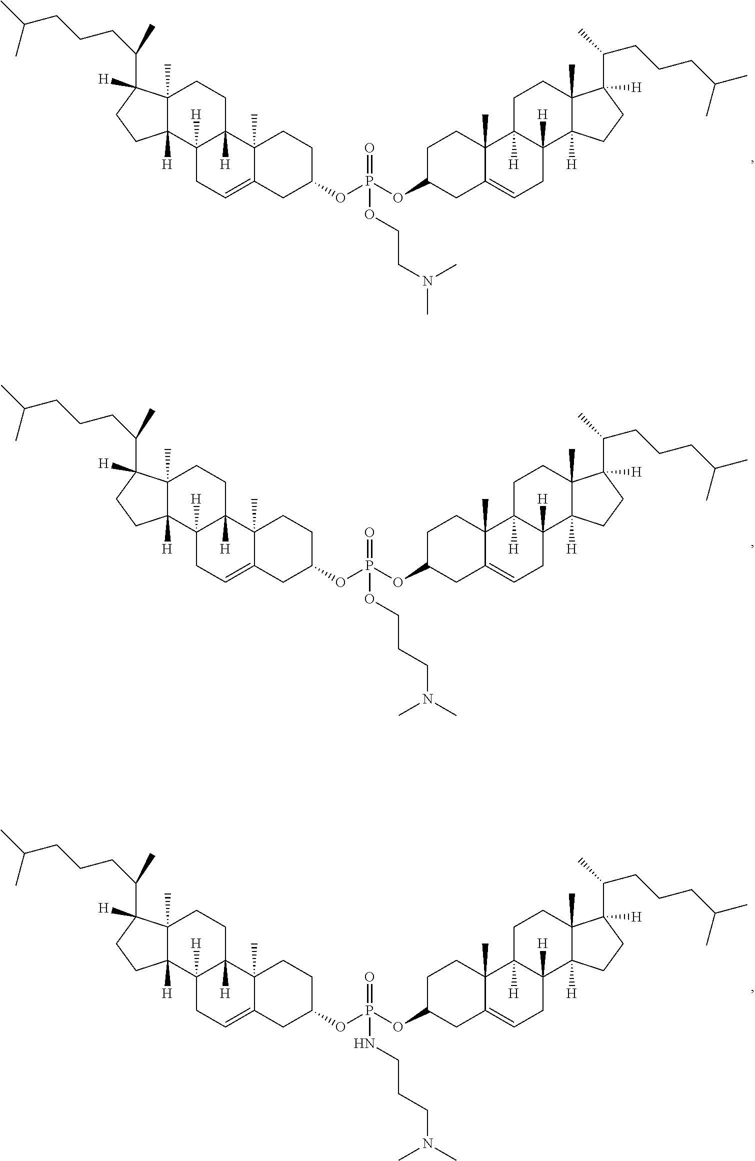

pharmaceutically acceptable salts thereof, and combinations thereof.

In some examples, the compound comprises:

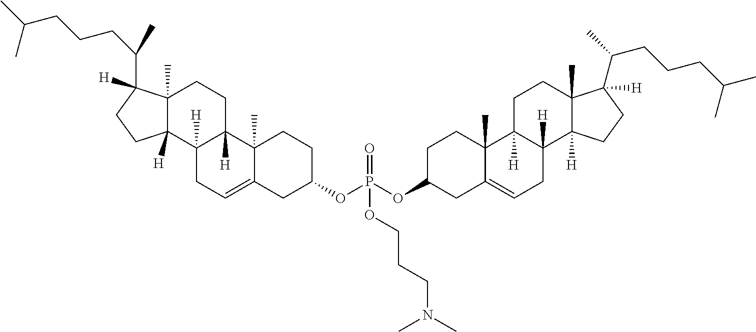

or a pharmaceutically acceptable salt thereof.

### Lipid Particle

Also disclosed herein is a lipid particle (e.g., one or more lipid particles) comprising any of the compositions or compounds disclosed herein.

The lipid particle can be of any shape. (e.g., a sphere, a rod, a quadrilateral, an ellipse, a triangle, a polygon, etc.). In some examples, the lipid particle can have a regular shape, an irregular shape, an isotropic shape, an anisotropic shape, or a combination thereof. In some examples, the lipid particle are substantially spherical in shape.

The lipid particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The lipid particles can, for example, have an average particle size of 30 nanometers (nm) or more (e.g., 40 nm or more, 50 nm or more, 60 nm or more, 70 nm or more, 80 nm or more, 90 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 375 nm or more, 400 nm or more, 425 nm or more, 450 nm or more, 475 nm or more, 500 nm or more, 550 nm or more, 600 nm or more, 650 nm or more, 700 nm or more, or 750 nm or more). In some examples, the lipid particles can have an average particle size of 800 nm or less (e.g., 750 nm or less, 700 nm or less, 650 nm or less, 600 nm or less, 550 nm or less, 500 nm or less, 475 nm or less, 450 nm or less, 425 nm or less, 400 nm or less, 375 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 90 nm or less, 80 nm or less, 70 nm or less, 60 nm or less, 50 nm or less, or 40 nm or less). The average particle size of the lipid particles can range from any of the minimum values described above to any of the maximum values described above. For example, the lipid particles can have an average particle size of from 30 nm to 800 nm (e.g., from 30 nm to 425 nm, from 425 nm to 800 nm, from 30 nm to 200 nm, from 200 nm to 400 nm, from 400 nm to 600 nm, from 600 nm to 800 nm, from 50 nm to 800 nm, from 30 nm to 750 nm, from 50 nm to 750 nm, from 50 nm to 500 nm, from 50 nm to 250 nm, from 100 nm to 200 nm, or from 100 nm to 150 nm).

With respect to particle size distribution characterization, a parameter used to define the size range of the lipid particles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the lipid particles can have a polydispersity index of 0.5 or less (e.g., 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the lipid particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

In some examples, the lipid particle can further comprise an additional component, such as an additional lipid. In some examples, the additional lipid can comprise a phospholipid, a sterol, or a combination thereof. In some examples, the lipid particle can further comprise 1,2-dioleoyl-sn-glycero-3-phosphoethanolamine (DOPE), cholesterol, 1,2-dimyristoyl-rac-glycero-3-methylpolyoxyethylene, or a combination thereof.

### Pharmaceutical Compositions

Also disclosed herein are pharmaceutical compositions comprising any of the compositions, compounds, and/or lipid particles disclosed herein.

For example, also disclosed herein are pharmaceutical compositions comprising a therapeutic agent encapsulated within any of the lipid particles disclosed herein. For example, the therapeutic agent can be encapsulated within the lipid particle with an encapsulation efficiency of 30% or more (e.g., 35% or more, 40% or more, 45% or more, 50% or more, 55% or more, 60% or more, 65% or more, 70% or more, 75% or more, 80% or more, 85% or more, 90% or more, 95% or more, or 99% or more).

The therapeutic agent can, for example, comprise an anticancer agent, an anti-inflammatory agent, an antimicrobial agent, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, bachimycin, halocyamine, hedamycin, helquinoline, berbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper (I) cyanide, copper (II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, S-hydroxy-2 (5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl) quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine, and combinations thereof.

In some examples, the therapeutic agent comprises an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C₂₂₅, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine,-Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent comprises a nucleic acid. Particular nucleic acid examples include, but are not limited to, oligonucleotides, miRNA, saRNA, shRNA, siRNA, DNA, RNA, mRNA, cDNA, double stranded nucleic acid, single stranded nucleic acid, and so forth. In some examples, the nucleic acid can be mRNA, saRNA, or a combination thereof. In some examples, the nucleic acid encodes a protein or peptide, e.g. for therapeutic use. In some examples, the nucleic acid encodes DNA Meiotic Recombinase 1 (Dmc1) protein.

In some examples, the pharmaceutical composition is administered to a subject. In some examples, the subject is a mammal. In some examples, the mammal is a primate. In some examples, the mammal is a human. In some examples, the human is a patient.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

### Methods of Making

Also disclosed herein are methods of making any of the compounds or compositions disclosed herein. Also disclosed herein are methods of making any of the lipid particles disclosed herein. Also disclosed herein are methods of making any of the pharmaceutical compositions disclosed herein.

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., 1H or 13C) infrared spectroscopy, spectrophotometry (e.g., UV-visible), or mass spectrometry, or by chromatography such as high performance liquid chromatography (HPLC) or thin layer chromatography.

### Methods of Use

Also disclosed herein are methods of use of any of the compounds, compositions, and/or lipid particles disclosed herein.

For example, also disclosed herein are methods of treating, preventing, or ameliorating a disease or a disorder in a subject in need thereof, the method comprising administering to the subject a therapeutically effective amount of any of the pharmaceutical compositions disclosed herein.

For example, disclosed herein are methods of treating a disease or a disorder in a subject in need thereof, the method comprising administering to the subject a therapeutically effective amount of any of the pharmaceutical compositions disclosed herein.

For example, the compounds and compositions described herein or pharmaceutically acceptable salts thereof are useful for treating a disease or disorder in humans, e.g., pediatric and geriatric populations, and in animals, e.g., veterinary applications. The disclosed methods can optionally include identifying a patient who is or may be in need of treatment of a disease or disorder.

In some examples, the subject is a human male and the disease or disorder comprises male infertility. In some examples, the method comprises delivering the therapeutically effective amount of the pharmaceutical composition to the testis. In some examples, the method comprises delivering the therapeutically effective amount of the pharmaceutical composition to spermatocytes.

The methods of treatment of the disease or disorder described herein can further include treatment with one or more additional agents. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

In some examples, the compound or composition can be administered to the subject in an amount of 1 microgram (μg) per kilogram (kg) of body weight of the subject per day (μg/kg/day) or more (e.g., 2 μg/kg/day or more, 3 μg/kg/day or more, 4 μg/kg/day or more, 5 μg/kg/day or more, 10 μg/kg/day or more, 15 μg/kg/day or more, 20 μg/kg/day or more, 25 μg/kg/day or more, 30 μg/kg/day or more, 35 μg/kg/day or more, 40 μg/kg/day or more, 45 μg/kg/day or more, 50 μg/kg/day or more, 60 μg/kg/day or more, 70 μg/kg/day or more, 80 μg/kg/day or more, 90 μg/kg/day or more, 100 μg/kg/day or more, 125 μg/kg/day or more, 150 g/kg/day or more, 175 μg/kg/day or more, 200 μg/kg/day or more, 225 μg/kg/day or more, 250 μg/kg/day or more, 300 μg/kg/day or more, 350 μg/kg/day or more, 400 μg/kg/day or more, 450 μg/kg/day or more, 500 μg/kg/day or more, 600 μg/kg/day or more, 700 μg/kg/day or more, 800 μg/kg/day or more, 900 μg/kg/day or more, 1 milligram (mg)/kg/day or more, 2 mg/kg/day or more, 3 mg/kg/day or more, 4 mg/kg/day or more, 5 mg/kg/day or more, 6 mg/kg/day or more, 7 mg/kg/day or more, 8 mg/kg/day or more, or 9 mg/kg/day or more). In some examples, the compound or composition can be administered to the subject in an amount of 10 milligrams (mg) per kilogram (kg) of body weight of the subject per day (mg/kg/day) or less (e.g., 9 mg/kg/day or less, 8 mg/kg/day or less, 7 mg/kg/day or less, 6 mg/kg/day or less, 5 mg/kg/day or less, 4 mg/kg/day or less, 3 mg/kg/day or less, 2 mg/kg/day or less, 1 mg/kg/day or less, 900 g/kg/day or less, 800 μg/kg/day or less, 700 μg/kg/day or less, 600 μg/kg/day or less, 500 μg/kg/day or less, 450 g/kg/day or less, 400 μg/kg/day or less, 350 μg/kg/day or less, 300 μg/kg/day or less, 250 μg/kg/day or less, 225 μg/kg/day or less, 200 μg/kg/day or less, 175 μg/kg/day or less, 150 μg/kg/day or less, 125 g/kg/day or less, 100 μg/kg/day or less, 90 μg/kg/day or less, 80 μg/kg/day or less, 70 μg/kg/day or less, 60 μg/kg/day or less, 50 μg/kg/day or less, 45 μg/kg/day or less, 40 μg/kg/day or less, 35 μg/kg/day or less, 30 μg/kg/day or less, 25 μg/kg/day or less, 20 μg/kg/day or less, 15 μg/kg/day or less, 10 μg/kg/day or less, 5 μg/kg/day or less, 4 μg/kg/day or less, 3 μg/kg/day or less, or 2 μg/kg/day or less).

The amount of the compound or composition administered to the subject can range from any of the minimum values described above to any of the maximum values described above. For example, the compound or composition can be administered to the subject in an amount of from 1 microgram (μg) per kilogram (kg) of body weight of the subject per day to 10 milligrams (mg)/kg/day (e.g., from 1 μg/kg/day to 100 μg/kg/day, from 100 μg/kg/day to 10 mg/kg/day, from 1 μg/kg/day to 10 μg/kg/day, from 10 μg/kg/day to 100 μg/kg/day, from 100 μg/kg/day to 1 mg/kg/day, from 1 mg/kg/day to 10 mg/kg/day, from 5 μg/kg/day to 10 mg/kg/day, from 1 μg/kg/day to 5 mg/kg/day, or from 5 to 5 mg/kg/day).

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

In certain embodiments, it is desirable to target a nanoparticle using a targeting moiety that is specific to a cell type and/or tissue type. In some embodiments, a nanoparticle may be targeted to a particular cell, tissue, and/or organ using a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′) 2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42 (5): 439-62, 2003 and Abra et al., J. Liposome Res. 12:1-3, 2002.

The targeting moiety can target any known cell type, including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells).

### Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically-or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as tale, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Cholesterol-Amino-Phosphate (CAP) Derived Lipid Nanoparticles for Delivery of Self-Amplifying RNA and Restoration of Mouse Spermatogenesis

Abstract. Male infertility caused by genetic mutations has become an important type of infertility. Currently, there is no reliable method in the clinic to address this medical need. The emergence of messenger RNA (mRNA) therapy provides a possible strategy for restoring mutant genes in the reproductive system. However, effective delivery of mRNA to spermatocytes remains a formidable challenge. Herein, it is shown that cholesterol-amino-phosphate (CAP) derived lipid nanoparticles (CAP LNPs) can deliver mRNA including traditional mRNA and self-amplifying RNA (saRNA) encoding DNA Meiotic Recombinase 1 (Dmc1) protein in spermatocytes and treat male infertility caused by the Dmc1 gene mutation. The results demonstrated that the delivery efficiency of CAP LNPs was significantly higher than that of the D-Lin-MC3-DMA LNPs. Importantly, the CAP LNPs-saRNA can produce Dmc1 protein for an extended period, which restored the Dmc1 function and spermatogenesis in the Dmc1 gene knockout (Dmc1-+) mouse model. This study illustrates the concept of LNPs for the delivery of mRNA to spermatocytes, which merits further development for the therapy of male infertility.

Results and Discussion. Male infertility accounts for approximately 50% of all infertility cases (Agarwal A et al. The Lancet, 2020, 397 (10271), 319-333). Currently, certain cases of male infertility can be treated with assisted reproductive techniques (ART) (Esteves SC. International Braz J Urol. 2020, 46, 116-123; Ramasamy R et al. Fertility and Sterility, 2009, 92, 590-593; Donoso P et al. Human Reproduction Update, 2007, 13, 539-549). However, ART cannot solve the issues of male infertility caused by genetic mutations (Miyamoto T et al. Journal of Obstetrics and Gynaecology Research, 2015, 41, 1501-1505; Simon A et al. Journal of Assisted Reproduction and Genetics, 2012, 29, 1227-1239). Common genetic mutations that cause male infertility in humans include synaptonemal complex protein 3 (Sycp3) gene and testis-expressed gene 11 (TEX11) mutations (Miyamoto T et al. The Lancet, 2003, 362, 1714-1719; Yatsenko A N et al. New England Journal of Medicine, 2015, 372, 2097-2107). These genetic mutations are usually associated with abnormal protein function in spermatocytes, meiotic arrest, and severe azoospermia (Dobson M J et al. Journal of Cell Science, 1994, 107, 2749-2760; Lammers J et al. Molecular and Cellular Biology, 1994, 14, 1137-1146; Yuan L et al. The Journal of Cell Biology, 1998, 142, 331-339; Yang F et al. Genes & Development, 2008, 22, 682-691; Tsubouchi T et al. Developmental Cell, 2006, 10, 809-819; Primig M et al. Nature Genetics, 2000, 26, 415-423). Unfortunately, there is no effective treatment in the clinic to restore the genetic function and fertility in humans. So far, genetic mutant animal models have been developed to study meiosis and gametogenesis in mammals. Similar to azoospermia in humans, it was reported that DNA Meiotic Recombinase 1 (Dmc1) gene knockout (Dmc1) mice showed complete absence of spermatogenesis (Pittman D L et al. Molecular Cell, 1998, 1, 697-705; Yoshida K et al. Molecular Cell, 1998, 1, 707-718). More importantly, mutation of Dmc1 gene can also lead to azoospermia by meiotic arrest since Dmc1 encodes DNA strand-exchange proteins which are important for the formation of homologous recombination (HR) and repairing DNA breaks during meiosis. Therefore, Dmc1^{-/-}mice are an appropriate therapeutic model for the development of new treatment strategies.

To address the important medical demand of male infertility, one of the challenges is to deliver therapeutic cargos into the diseased cell population such as spermatocytes. Lipid and lipid-derived nanoparticles (LNPs) are one of the most widely used nanomaterials for mRNA delivery owing to their low immunogenicity and favorable delivery efficiency (Li B et al. Wiley Interdisciplinary Reviews: Nanomedicine and Nanobiotechnology, 2019, 11, e1530; Hajj K A et al. Nature Reviews Materials, 2017, 2, 1-17; Kowalski P S et al. Molecular Therapy, 2019, 27, 710-728). In 2020, two LNP-messenger RNA (mRNA) vaccines have been applied for emergency use against severe acute respiratory syndrome coronavirus 2 (SARS-COV-2) (Baden L R et al. New England Journal of Medicine, 2021, 384, 403-416; Polack F P et al. New England Journal of Medicine, 2020, 383, 2603-2615). Meanwhile, researchers have investigated LNP-mRNA formulations for a variety of therapeutic applications, such as genetic engineering and protein replacement therapy (Pardi N et al. Nature Communications, 2017, 8, 1-8; Finn J D et al. Cell Reports, 2018, 22, 2227-2235; Akinc A et al. Nature Nanotechnology, 2019, 14, 1084-1087). Although LNPs have served as an important delivery system in various tissues including liver, lung, spleen, and tumor (Zhang X et al. Science Advances, 2020, 6, eabc2315; Zhang C et al. Nano Research, 2019, 12, 855-861; Cheng Q et al. Nature Nanotechnology, 2020, 15, 313-320; Kaczmarek J C et al. Nano Letters, 2018, 18, 6449-6454), effective delivery in testis especially spermatocytes remains a formidable challenge and an underexplored field for mRNA delivery.

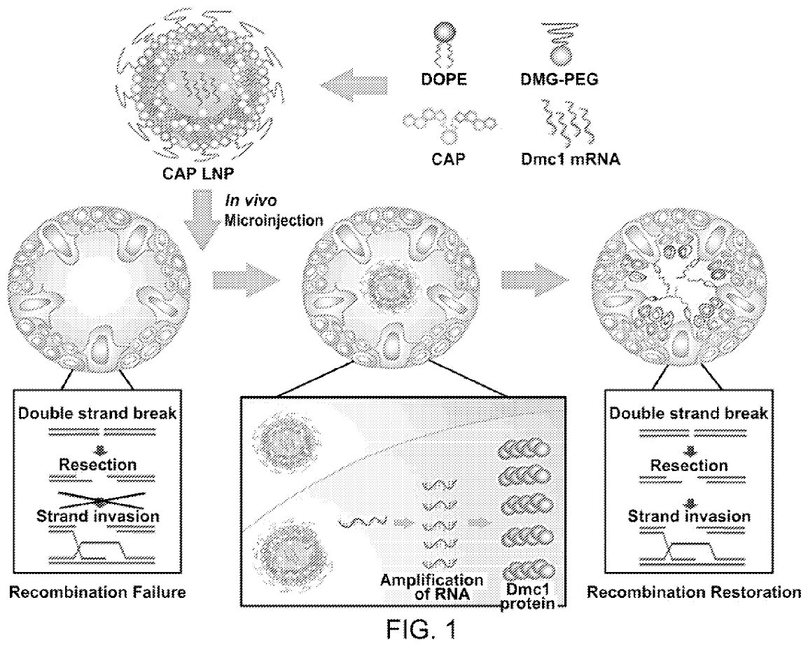

To facilitate the delivery of mRNA to spermatocytes, some bioactive molecules attracted attention for this study due to their important roles in biological membranes and the male reproductive system. For example, cholesterol is an essential structural component of mammalian cell membranes, which maintain the integrity and permeability of membranes. Prior studies have also reported elevated cholesterol levels during spermatogenesis, indicating a close relationship between cholesterol metabolism and fertility (Sèdes L et al. Frontiers in Endocrinology, 2018, 9, 369; Potter J et al. Journal of Biological Chemistry, 1981, 256, 7150-7154). Additionally, the phosphate group is a polar part of the phospholipid which is a key component of biological membranes and participates in cell transport pathways. Lastly, amino groups can be ionized to become positively charged, which can interact with negatively charged mRNA. By integrating these three units, a series of cholesterol-amino-phosphate (CAP) derivatives were designed, which can be formulated to CAP LNPs (FIG. 1). After microinjection into seminiferous tubules, CAP LNPs can release mRNAs to the cytosol, which are translated to Dmc1 proteins. The sufficient supplementation of Dmc1 protein can rescue the chromosomes recombination, thereby recovering the meiosis and spermatogenesis.

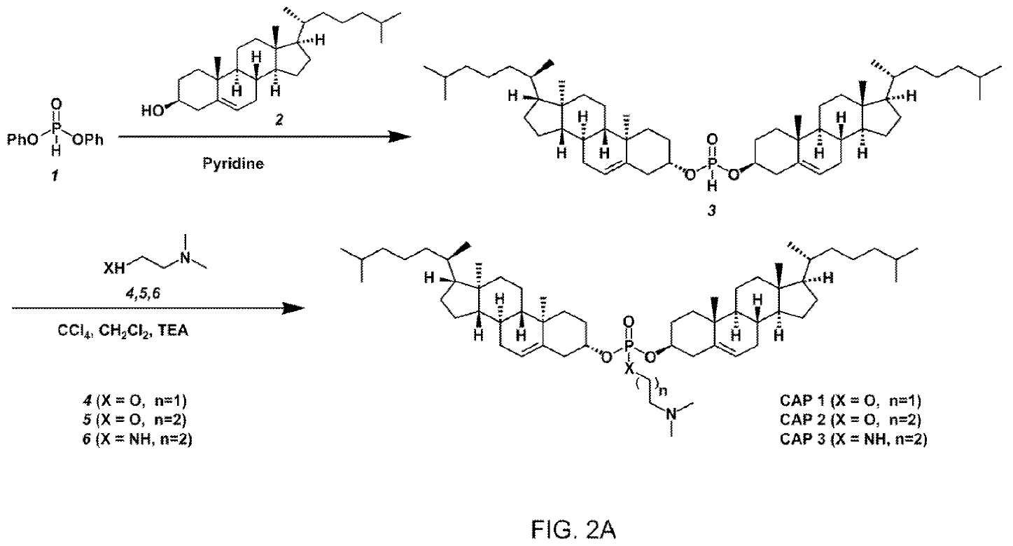

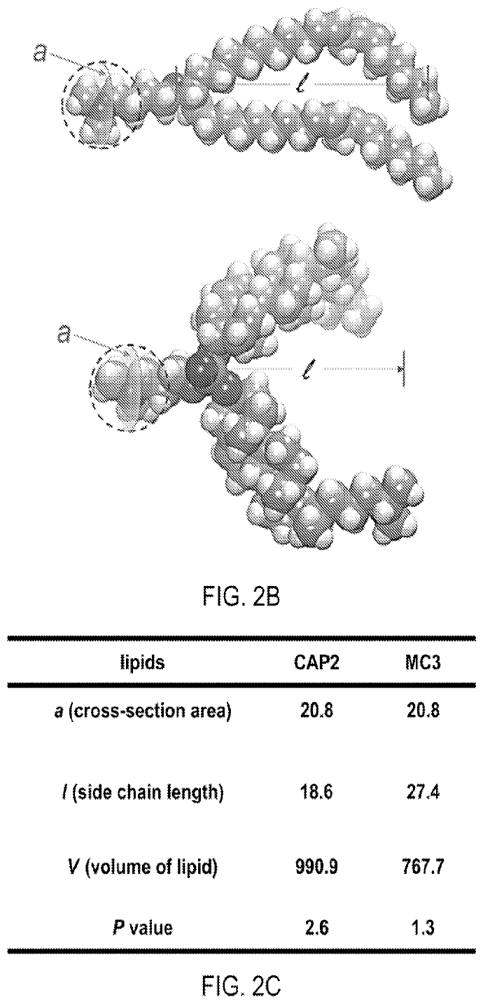

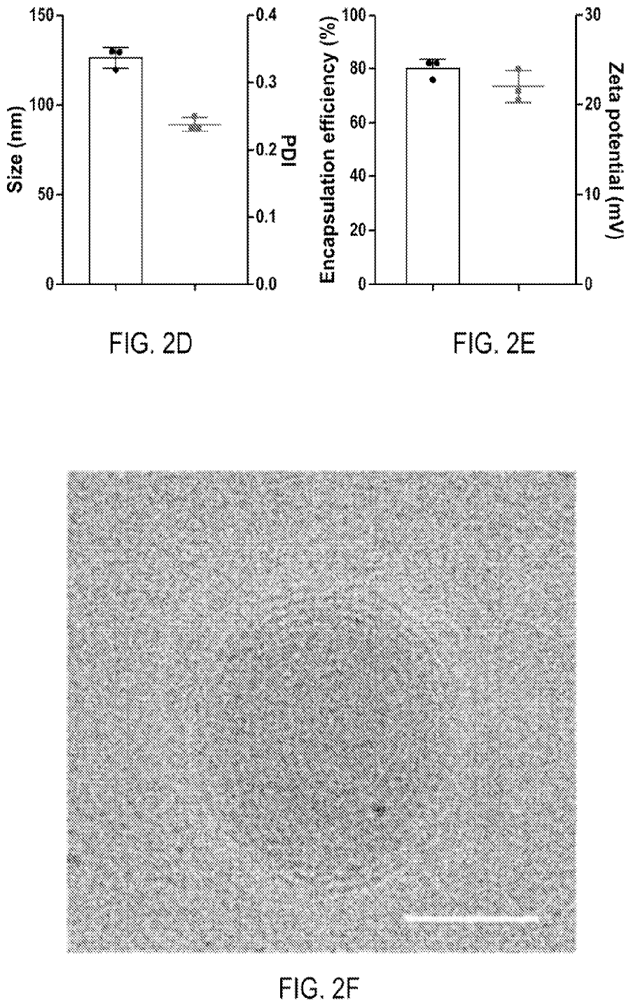

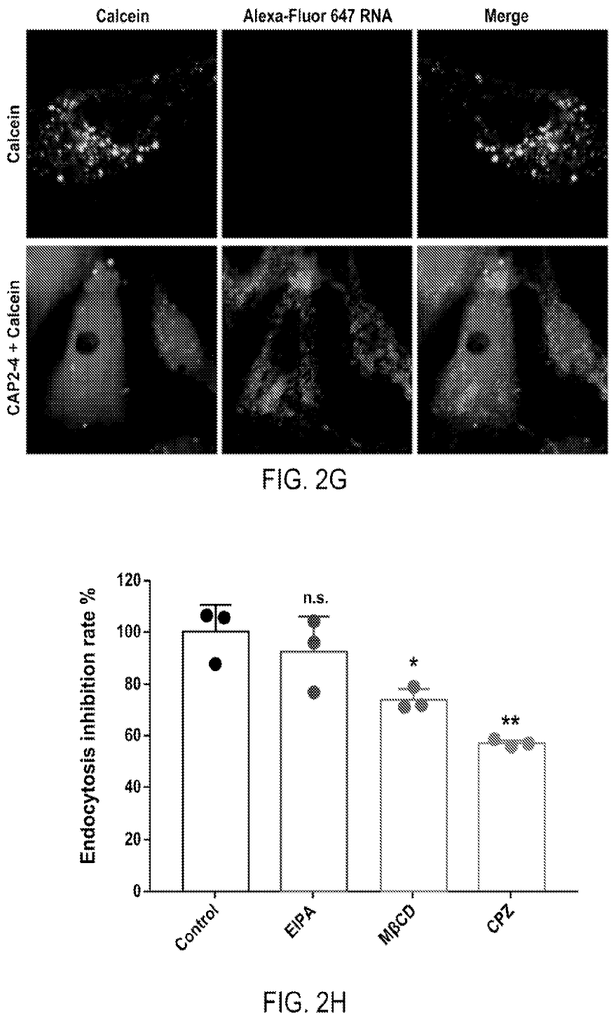

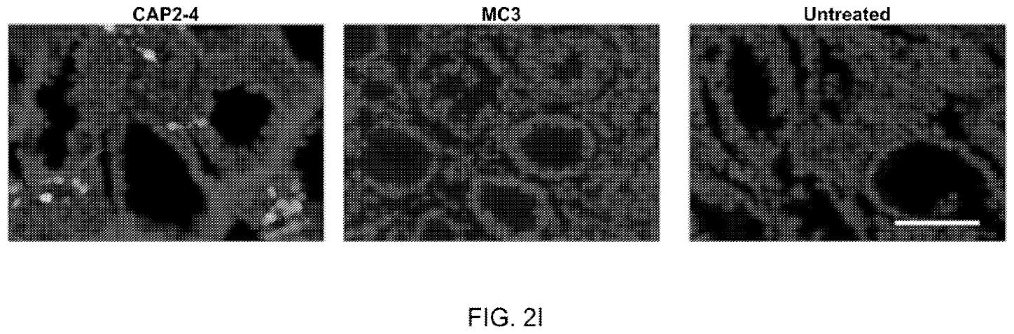

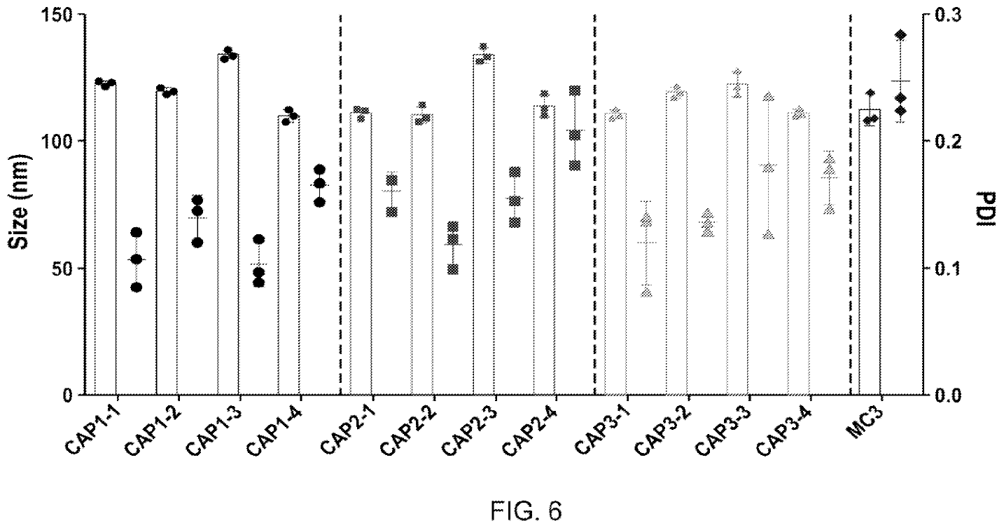

First, a synthetic route was constructed to cholesterol-amino-phosphate (CAP) derivatives (FIG. 2A), which include two cholesterols, one phosphate linker, and an amino group. Briefly, dicholesterol phosphite (compound 3) was obtained through a transesterification reaction of diphenyl phosphite and cholesterol. Compound 3 then underwent an Atherton-Todd reaction with amines or amino alcohols in the presence of carbon tetrachloride to afford the corresponding phosphoramidate or phosphotriester, respectively (Atherton F et al. Journal of the Chemical Society (Resumed), 1945, 0, 660-663). Their structures were then confirmed by H NMR and mass spectrum (MS). Next, these CAP lipids were formulated with Dioleoyl phosphatidylethanolamine (DOPE), cholesterol, 1,2-dimyristoyl-rac-glycero-3-methoxypolyethylene glycol-2000 (DMG-PEG2000), and firefly luciferase mRNA to prepare CAP LNPs (Table 1) (Zhang X et al. Science Advances, 2020, 6, eabc2315; Hou X et al. Nature Nanotechnology 2020, 15, 41-46). CAPs LNPs showed comparable particle size from 100 nm to 150 nm with PDI lower than 0.3 (FIG. 6).

TABLE 1
Formulation of the CAP LNPs for in vitro luciferase assay.

| Name | CAP lipid | DOPE | Cholesterol | DMG-PEG |
|---|---|---|---|---|
| CAP1-1 | 20 | 30 | 40 | 0.75 |
| CAP1-2 | 20 | 30 | 0 | 0.75 |
| CAP1-3 | 60 | 30 | 40 | 0.75 |
| CAP1-4 | 60 | 30 | 0 | 0.75 |
| CAP2-1 | 20 | 30 | 40 | 0.75 |
| CAP2-2 | 20 | 30 | 0 | 0.75 |
| CAP2-3 | 60 | 30 | 40 | 0.75 |
| CAP2-4 | 60 | 30 | 0 | 0.75 |
| CAP3-1 | 20 | 30 | 40 | 0.75 |
| CAP3-2 | 20 | 30 | 0 | 0.75 |
| CAP3-3 | 60 | 30 | 40 | 0.75 |
| CAP3-4 | 60 | 30 | 0 | 0.75 |

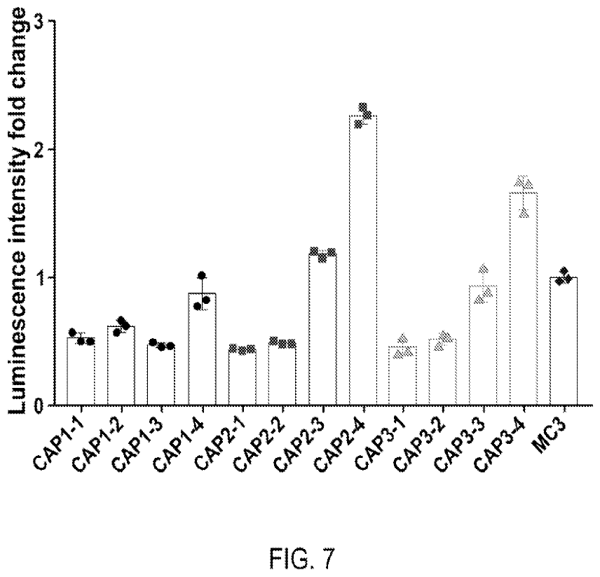

Then, the mRNA delivery efficiency of CAP LNPs was investigated in Hep3B cells in vitro. D-Lin-MC3-DMA also known as MC3, is an FDA-approved ionizable amide lipid that has been used as a lipid component in ONPATTROR. In this study, MC3 based LNPs were included as a control group. CAP2-4 LNPs showed the highest luminescence intensity among all the formulations (FIG. 7), which was around twice the luminescence intensity of MC3 LNPs.

The geometry of the ionizable lipids, which can be described by packing parameter (P value), can greatly affect the delivery efficiency of the LNPs. Typically, lipids with a large P value are favorable to form an inverted conical shape, which facilitates the inverted hexagonal (Hn phase) transformation of the endosome membrane and the endosomal escape of payloads. Hence, the P value of CAP2 and MC3 was calculated, which can predict the nanostructure formed by the lipids. In general, lipids of small P values (<⅓) would aggregate into spheres. Lipids of medium P values would pack into hexagonal (Hr phase) (⅓≤P≤½) or planar (½<P≤1) nanostructures. Large P values (>1) that correspond to “a small head with large tails” would lead to Hu phase or reversed spherical nanostructures. P value for a lipid molecule is calculated as: P=v/(la), where a is head area, and v and l are the tail volume and length, respectively. The calculated P value of CAP2 is 2.6, which is much higher than that of MC3 (P=1.3) (FIG. 2B, FIG. 2C). In general, the formation of the Hu phase is thought to be more likely to induce the rupture of the endosomal membrane and the release of RNA payloads into cytosol. Therefore, CAP2 with a P value of 2.6 is more likely to assemble into a Hn phase nanostructure and facilitate the intracellular mRNA delivery, which supports our design of the CAP molecules.

Based on these results, CAP2-4 LNPs were selected for the following studies and further characterized their physicochemical properties. The size of CAP2-4 LNPs was 126.4±5.7 nm with a PDI of 0.24±0.01 (FIG. 2D, FIG. 2E). CAP2-4 LNPs displayed a slightly positive charge, and the mRNA encapsulation efficiency was around 90%. Cryo-electron microscopy (cryo-EM) showed that CAP2-4 LNPs were spherical and multi-layered particles with a size around 100 nm, which was consistent with the Dynamic Light Scattering (DLS) measurement (FIG. 2F). A calcein assay was conducted to visualize the endosomal escape of CAP2-4 LNPs. Calcein is a membrane-impermeable fluorescent indicator that is normally trapped in endosomes. Upon permeabilization of the endosomal membrane, released calcein showed a diffuse fluorescent signal, indicating endosomal escape. In the experiment, diffused green fluorescence was observed in the cytoplasm when Hep3B cells were treated with both calcein and CAP2-4 LNPs. In the control group with only calcein incubated cells, the fluorescent signals were shown in scattered dots. These results suggest the rupture of endosomal compartments following CAP2-4 LNPs treatment (FIG. 2G). To study the endocytic pathway of CAP2-4 LNPs, Hep3B cells were incubated with endocytosis inhibitors including 5-(N-Ethyl-N-isopropyl) amiloride (EIPA), chlorpromazine (CPZ), and methyl-β-cyclodextrin (MBCD) to inhibit the macropinocytosis, clathrin, and caveolae endocytic pathways, respectively. CAP2-4 LNPs encapsulated with Alexa-Fluor-647-labeled RNA were then added to the pre-treated cells. It was found that the cellular uptake of CAP2-4 LNPs was significantly reduced by 26% and 43% after incubation by MBCD and CPZ, respectively, indicating that the uptake of CAP2-4 LNPs is mediated by both clathrin and caveolae pathways tested in this study (FIG. 2H).

CAP2-4 LNPs were applied to encapsulate the green fluorescent protein (GFP) mRNA as a reporter and evaluated its delivery efficiency in a Dmc1″-mouse model. CAP2-4 LNPs were microinjected to seminiferous tubules and expression of GFP was analyzed 24 h after administration. MC3 LNPs were administered in the same way as a control. As shown in FIG. 2I, CAP2-4 LNPs induced dramatic green fluorescence around the seminiferous tubules, while the GFP signal in MC3 LNPs group was barely detected. These results demonstrated that CAP2-4 LNPs were superior vehicles for mRNA delivery in spermatocytes.

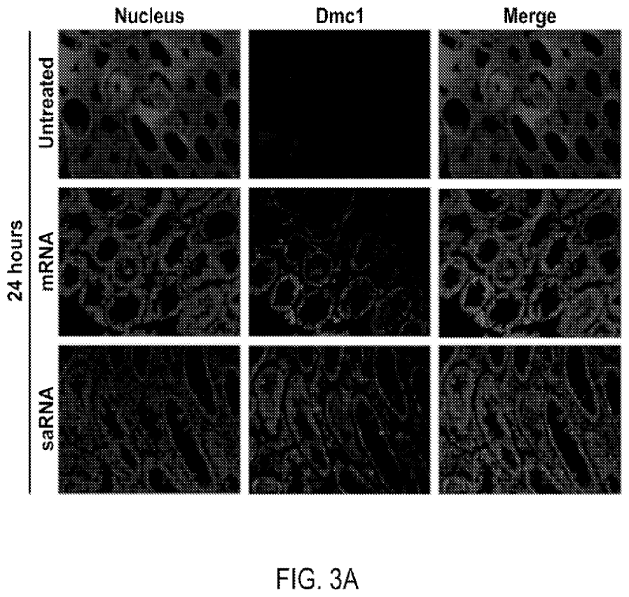

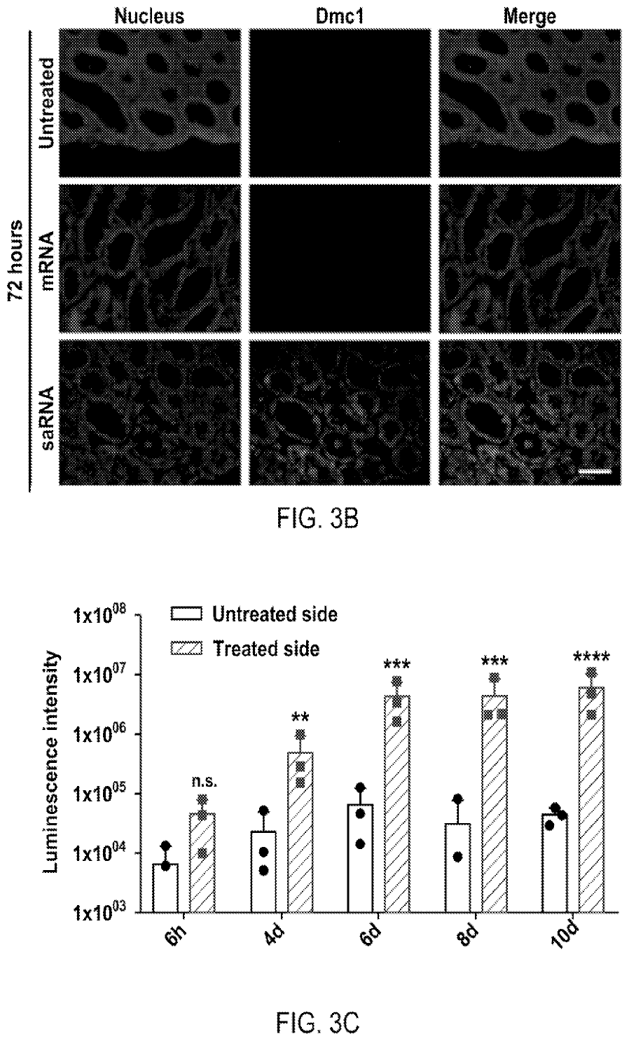

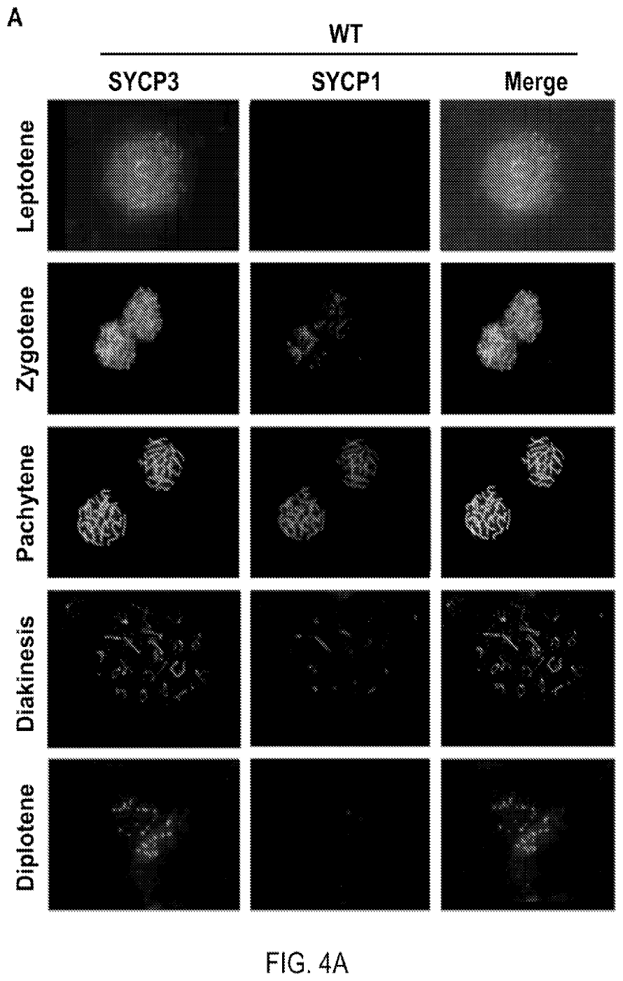

Unlike traditional mRNA, self-amplifying mRNA (saRNA, also named replicon RNA) encodes not only the proteins of interest but also proteins that enable intracellular RNA amplification (Vogel A B et al. Molecular Therapy 2018, 26, 446-455). Thus, saRNA can induce the expression of desired proteins for a longer period compared to traditional mRNA (Bloom K et al. Gene Therapy, 2020, 28, 1-13). To validate the ability of CAP LNPs to deliver saRNA in vivo, firefly luciferase saRNA was encapsulated in CAP2-4 LNPs and injected into seminiferous tubule of Dmc1^{-/-}mice. As shown in FIG. 3C, the luminescence intensity on the injected side (left testis) continued to increase for 10 days, demonstrating that CAP2-4 LNPs can efficiently deliver saRNA into testis and achieve sustained protein expression. The expression time course of Dmc1 protein was then compared between traditional mRNA and saRNA. Flag-tagged Dmc1 mRNA or saRNA was encapsulated in CAP2-4 LNPs and both formulations were injected into seminiferous tubule of Dmc1^{-/-}mice. On day 1 (24 hours) and day 3 (72 hours) after administration, Dmc1 expression in seminiferous tubules was evaluated by immunofluorescence staining of the Flag tag. As shown in FIG. 3A and FIG. 7, obvious Dmc1 expression was observed in both CAP2-4 LNP-mRNA and CAP2-4 LNP-saRNA treated mice on day 1 but not in untreated mice. On day 3 after the administration of the CAP2-4 LNP-mRNA, there was almost no fluorescence signal in the seminiferous tubules (FIG. 3B). On the contrary, the CAP2-4 LNP-saRNA-treated group still displayed a strong fluorescence signal (FIG. 3B). These results indicated that the delivery of saRNA via CAP2-4 LNP can maintain the protein expression for at least three days. In male mice, it takes around 3 days from the initiation of prophase I to pachytene spermatocytes. During this period, Dmc1 is required for chromosome recombination (Oakberg EF. American Journal of Anatomy 1956, 99, 507-516), thus the formulation of CAP2-4 LNP-saRNA was selected for further functional studies.

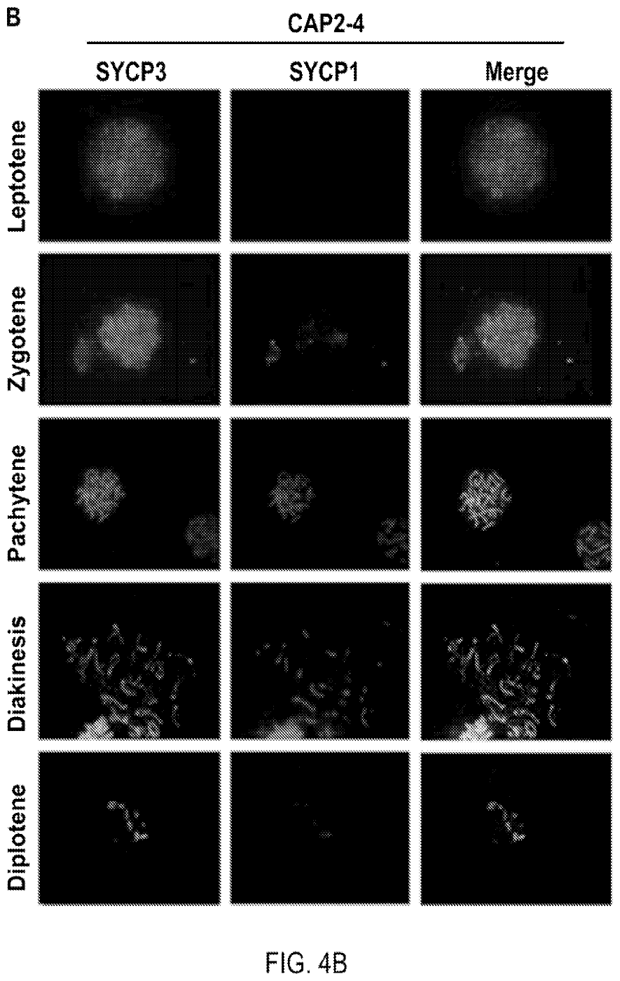

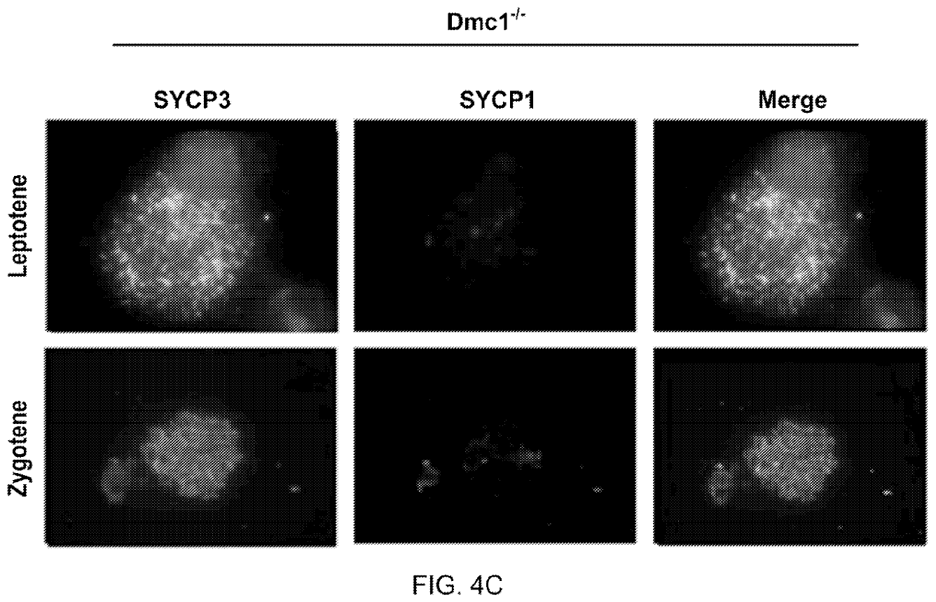

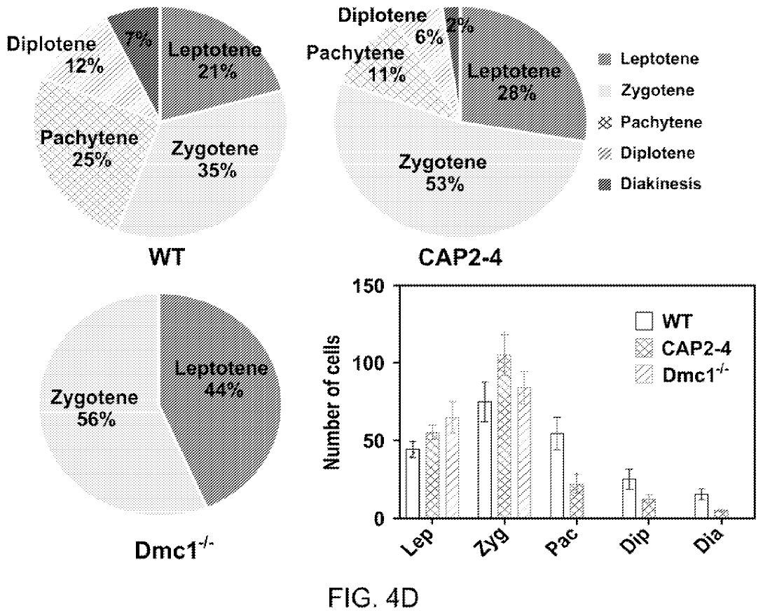

Subsequently, the function of Dmc1 protein was examined by analyzing synaptonemal complex (SC). SC is a specific structure formed during meiosis I and is responsible for the synapsis of homologous chromosomes. Since Dmc1 deficiency can disrupt the SC formation and arrest the spermatogenesis in the late prophase of Meiosis I, SC is considered a critical indicator of Dmc1 protein function recovery (Sehorn M G et al. Nature 2004, 429, 433-437; Hong E L et al. Journal of Biological Chemistry 2001, 276, 41906-41912; Tarsounas M et al. Journal of Cell Biology 1999, 147, 207-220). In this study, the Dmc1^{-/-} mice were treated with CAP2-4 LNP-saRNA and their testes were harvested on day 4 after treatment. The SC in the spermatocytes was examined by immunofluorescence staining of SYCP1 and SYCP3 (two main constitutes involved in SC formation). As shown in FIG. 4A, SYCP3 and SYCP1 staining clearly showed the characteristic patterns of the five substages including leptotene, zygotene, pachytene, diplotene, and diakinesis in WT mice (Dia F et al. Journal of Visualized Experiments: JoVE, 2017, 129, e55378; Holloway J K et al. The Journal of Cell Biology 2014, 205, 633-641). In contrast, the SC from Dmc1^{-/-}mice was arrested in the early stage of prophase and the SC of pachytene, diplotene, and diakinesis stages was not observed (FIG. 4C). In the CAP2-4 LNP-saRNA treated group, the SC was observed in the pachytene stage which indicated the restoration of synapsis. In addition, the observation of SC in the diplotene and diakinesis stages further confirmed that SC can disassemble and transit to metaphase (FIG. 4B). As indicated by FIG. 4D, the percentages of spermatocytes in the stages of pachytene, diplotene, and diakinesis increased to 11%, 6%, and 2%, respectively after being treated by CAP2-4 LNP-saRNA. The number of SC in the last three substages (pachytene, diplotene, and diakinesis stages) was significantly higher than Dmc1^{-/-}mice. Collectively, these results demonstrated that the delivery of saRNA using CAP LNPs restored the function of Dmc1 protein in spermatocytes.

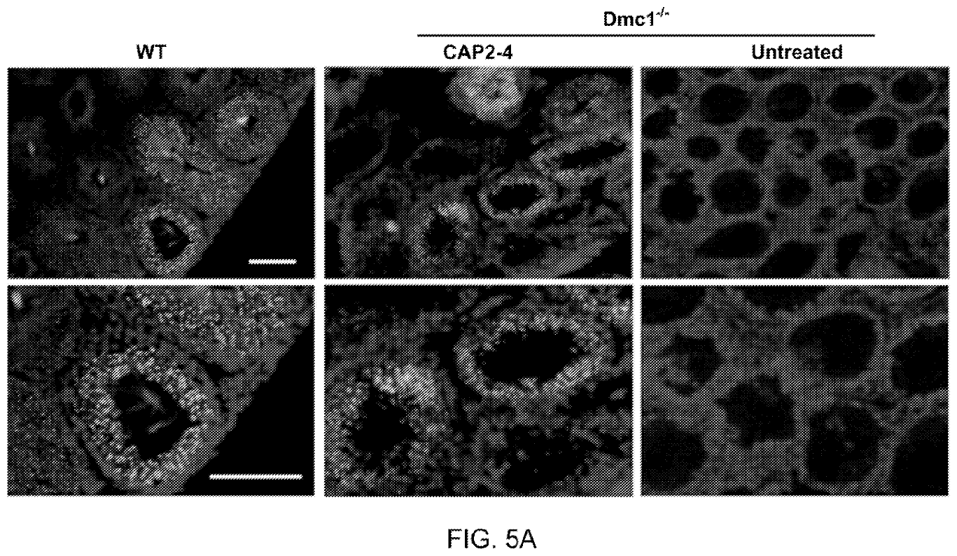

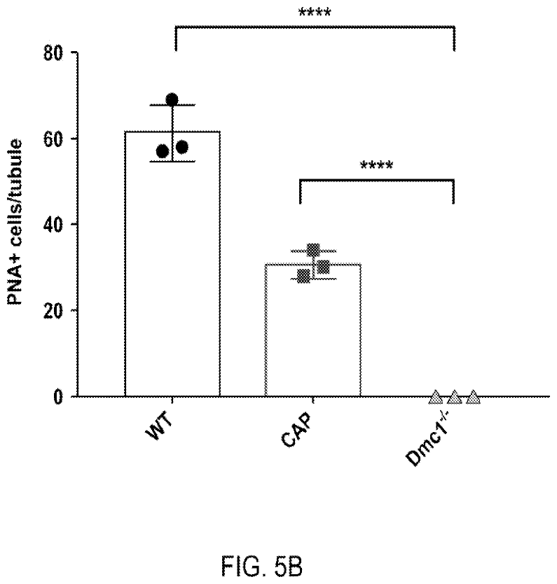

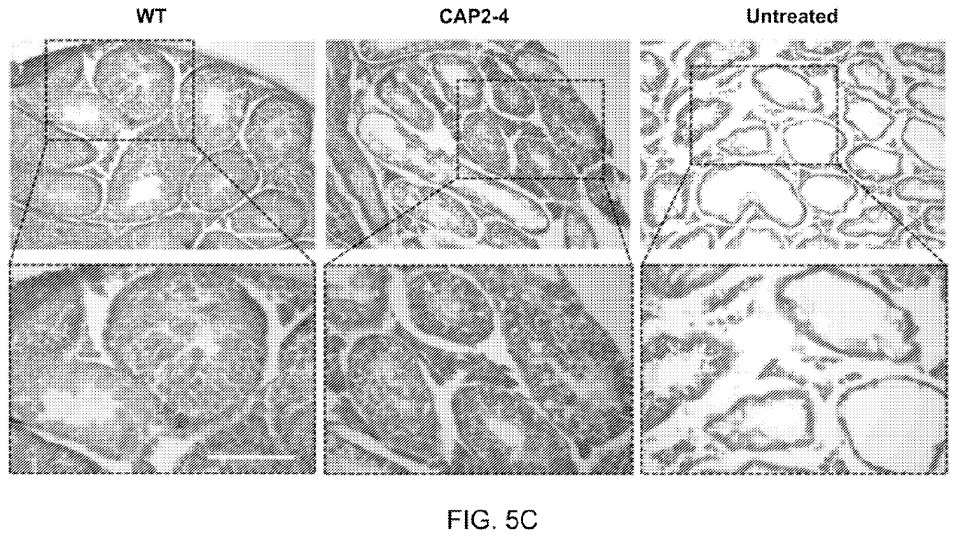

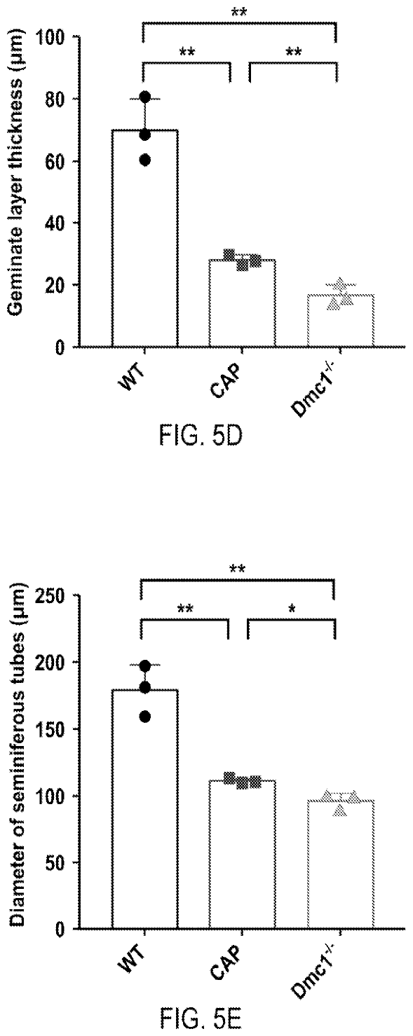

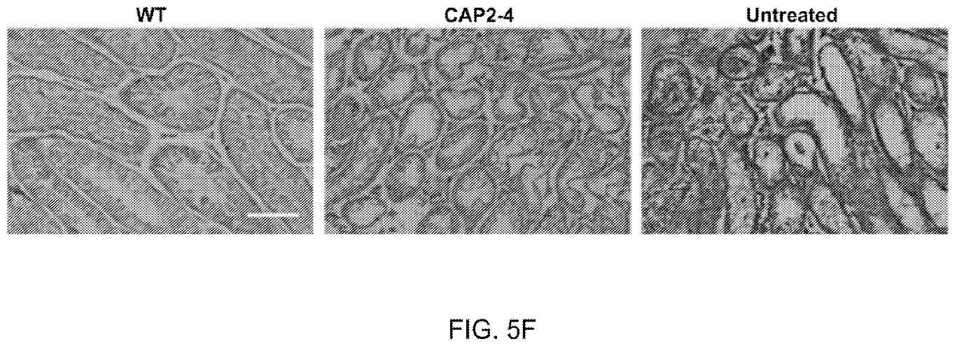

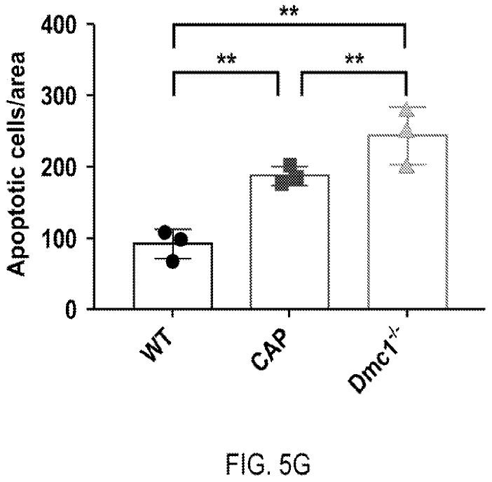

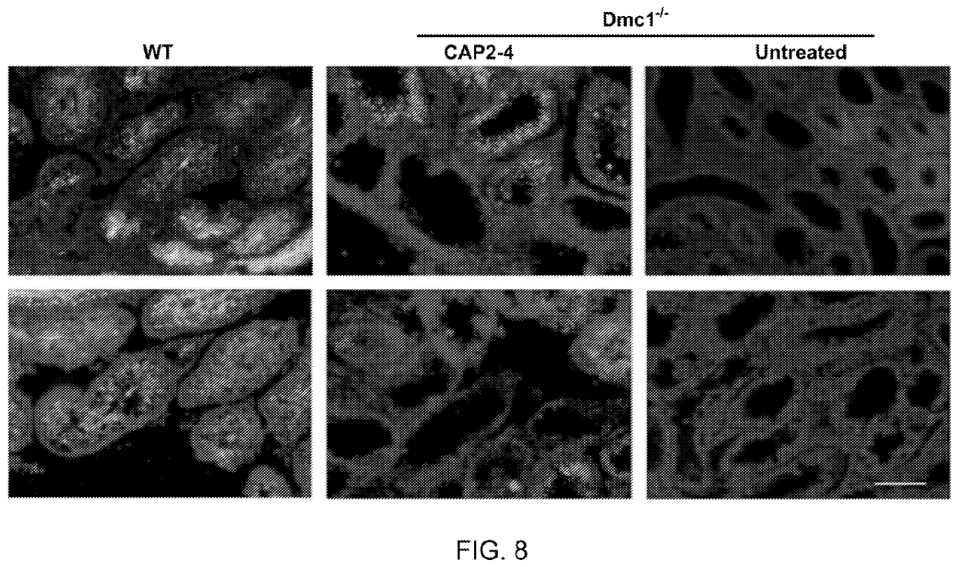

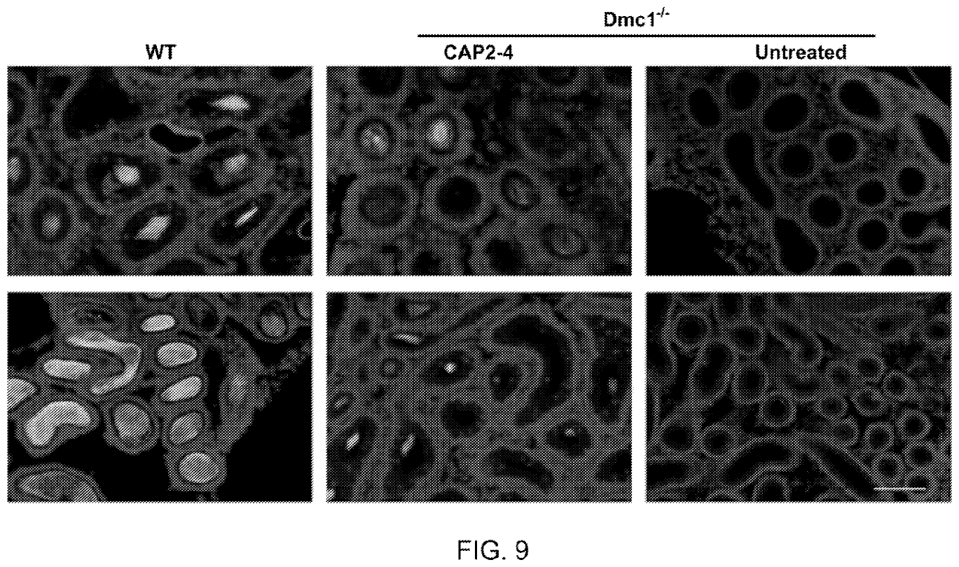

Finally, whether the restoration of Dmc1 protein by CAP2-4 LNP-saRNA can rescue the infertile phenotype of Dmc1^{-/-}mice was investigated. To directly visualize the spermatids' production, immunofluorescence analysis was performed on Dmc1 mice 10 days after being treated by CAP2-4 LNP-saRNA. The sections of testes and epididymis were stained with Peanut Agglutinin (PNA)-lectin which can specifically bind to spermatids acrosomes (FIG. 5A, FIG. 8, FIG. 9). PNA-stained spermatids were localized in the seminiferous tubules and epididymal ducts of WT mice. However, no cells in the testes and epididymis of Dmc1^{-/-}mice were stained with PNA-lectin, indicating a complete loss of spermatids production. In contrast, the PNA-stained cells were observed in the seminiferous tubules and epididymal ducts in the CAP2-4 LNP-saRNA treatment group. The statistical analysis showed that the number of PNA-positive cells per tubule in the treatment group was significantly higher than that in the untreated group and reached about one-half of the WT group (FIG. 5B). Therefore, these results showed that delivery of saRNA using CAP LNPs can effectively rescue spermatids production in Dmc1^{-/-}mice.

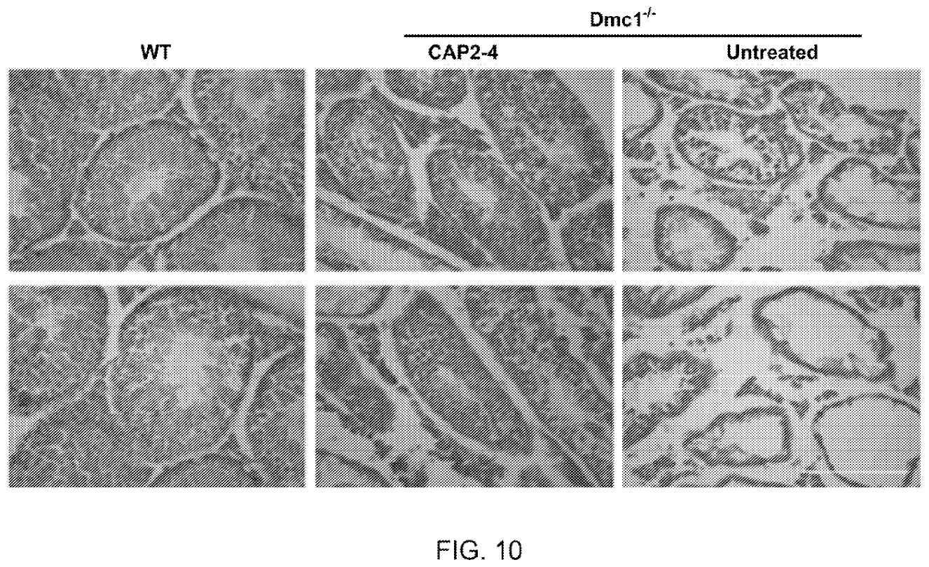

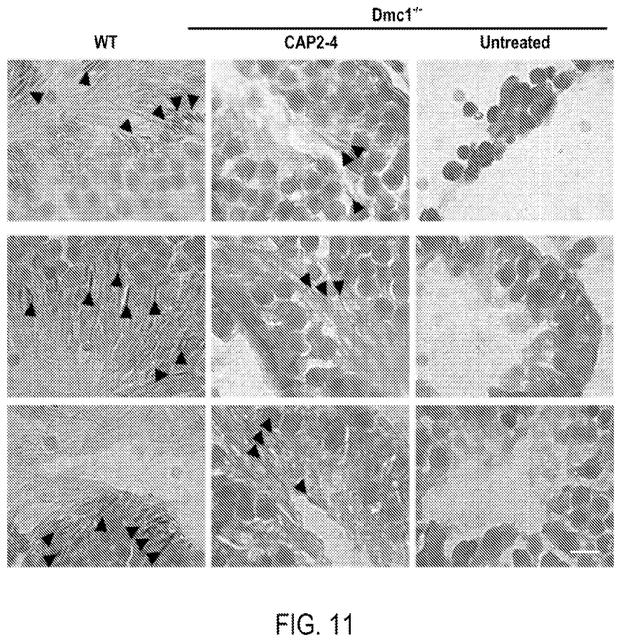

Additionally, histological analysis of testis samples was performed to evaluate spermatogenic development. As shown in Figure SC and FIG. 10, WT mice exhibited normal testicular morphology, while Dmc1-mice displayed an arrest of spermatogenesis at the spermatocyte stage and completely lacked post-meiotic cells. Compared with WT mice, the thickness of the germinal layer and the diameter of the seminiferous tubes were significantly reduced in Dmc1^{-/-}mice (FIG. 5D and Figure SE). In contrast, CAP2-4 LNP-saRNA re-establish the testicular morphology of Dmc1^{-/-}mice with increased germinate layer thickness and diameter of seminiferous tubes (FIG. 5D and FIG. 5E). At a high magnification (100×), cells were observed in the post-meiotic stage in the CAP2-4 LNP-saRNA treated group and their structure is similar to mature spermatids of WT mice (FIG. 11).

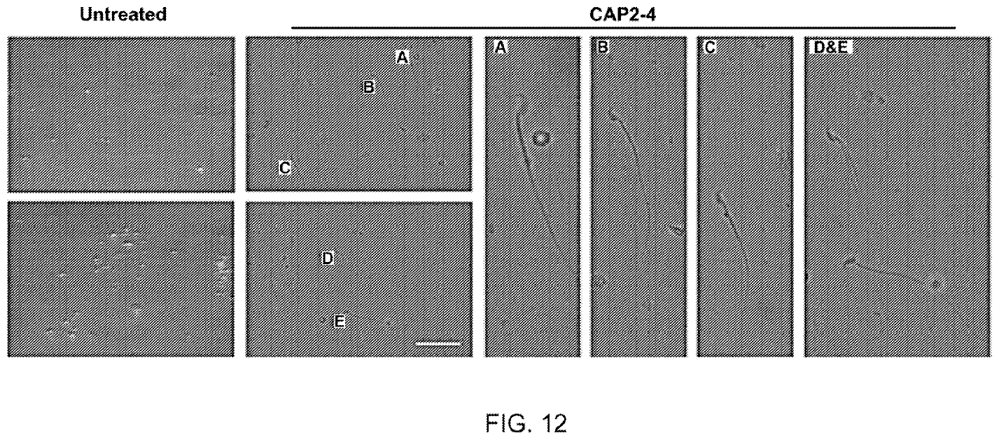

Another phenotype of Dmc1 deficiency is the abnormal apoptosis of spermatocytes; thus a TUNEL assay was conducted to study the apoptosis in different groups. FIG. 5F showed that compared with Dmc1^{-/-}mice, the apoptotic cells in the testes of CAP2-4 LNPs treated mice were reduced. Statistical analysis further confirmed that CAP2-4 LNP-saRNA significantly decreased the apoptosis of spermatocytes in Dmc1^{-/-}mice (FIG. 5G). The epididymis from CAP2-4 LNP-saRNA treated and untreated Dmc1^{-/-}mice was also incubated in Human Tubal Fluid (HTF) medium for 20 minutes to release sperms. The cytology smears of the sperm suspension showed that the epididymis of the CAP2-4 LNP-saRNA treated group produced sperms with oval-shaped head and an uncurled single tail, while no sperm-like cell was observed in the untreated group (FIG. 12). Taken together, these results indicated that delivery of saRNA using CAP LNPs can rescue the infertile phenotype of Dmc1^{-/-}mice. Considering the severity of infertility caused by Dmc1 deficiency, the restoration of the infertile phenotype of Dmc1^{-/-}mice validated the effectiveness of CAP LNPs to deliver saRNA in spermatocytes. In particular, the production of spermatids highlights the potential of this therapeutic strategy in the treatment of male infertility caused by genetic mutations.

In summary, CAP LNPs were designed and developed to facilitate the delivery of mRNA to spermatocytes and treat male infertility. Interestingly, a formulation of CAP LNPs formulated from three components (CAP, DOPE, and DMG-PEG) without the extra addition of cholesterol was identified, which simplified the nanoparticle composition. More importantly, it was shown that by using the newly developed CAP LNP to deliver the saRNA encoding the Dmc1 protein, the functional protein was restored and the sterility phenotype in the Dmc1″ mouse model was reversed. This is the first time saRNA has been applied for treating genetic diseases other than immunotherapy. Overall, this work demonstrates the feasibility of mRNA-based therapy in the treatment of male reproductive disorders caused by genetic mutations. This LNPs-saRNA system can be further developed and used clinically as a complement of ART to benefit a wide range of infertility.

### Methods

Materials and reagents. DOPE, cholesterol, and DMG-PEG2000 were purchased from Avanti Polar Lipids Inc. (Alabaster, AL). Eagle's minimum essential medium (EMEM) and other cell culture supplies were purchased from Corning Incorporated (Corning, NY). Quant-iT RiboGreen RNA reagent and Gibco heat-inactivated fetal bovine serum (FBS) were purchased from Thermo Fisher Scientific (Waltham, MA). All the other chemical reagents were obtained from Sigma-Aldrich or Abcam and used without further purifications.

Preparation and characterization of CAPs

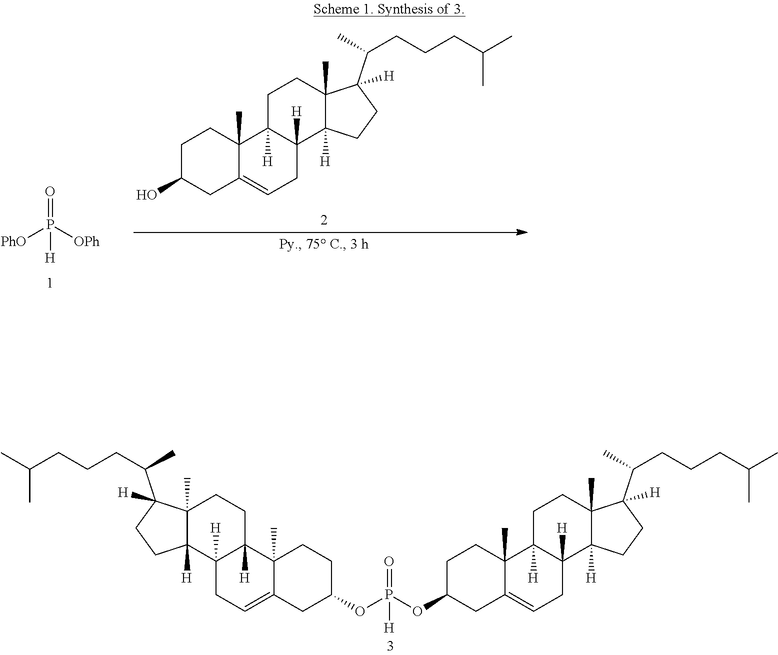

Synthesis of 3. Cholesterol 2 (2.38 g, 6.15 mmol) was added to a solution of diphenyl phosphonate 1 (0.7 g, 3.0 mmol) in 3.0 mL of pyridine. The resulting solution was then allowed to warm to 75° C. and stirred for 3 h. Pyridine was removed under reduced pressure, the residue was diluted with 100 mL of DCM and washed with 10 mL of 1 N aqueous NaOH solution and 10 mL of water. The organic phase was dried over anhydrous Na₂SO₄, filtered, and the solvent was removed under reduced pressure. The residue was purified by silica gel chromatography (0%-10% Ethyl acetate in Hexane). 2.0 g of compound 3 was obtained as a white powder, yield 81.4%. ¹H NMR (300 MHz, Chloroform-d) δ 6.89 (d, J=688.8 Hz, 1H), 5.38 (d, J=5.1 Hz, 2H), 4.27 (ddt, J=15.3, 10.8, 5.4 Hz, 2H), 2.52-2.38 (m, 4H), 1.98 (ddd, J=13.5, 6.9, 2.7 Hz, 6H), 1.89-1.79 (m, 4H), 1.78-1.66 (m, 2H), 1.58 (dd, J=7.2, 4.5 Hz, 2H), 1.55-1.40 (m, 10H), 1.39-1.23 (m, 8H), 1.23-1.04 (m, 14H), 1.04 (s, 2H), 1.01 (s, 6H), 0.97 (d, J=1.9 Hz, 2H), 0.93 (s, 2H), 0.91 (d, J=6.6 Hz, 6H), 0.86 (dd, J=6.6, 1.5 Hz, 12H), 0.67 (s, 6H).

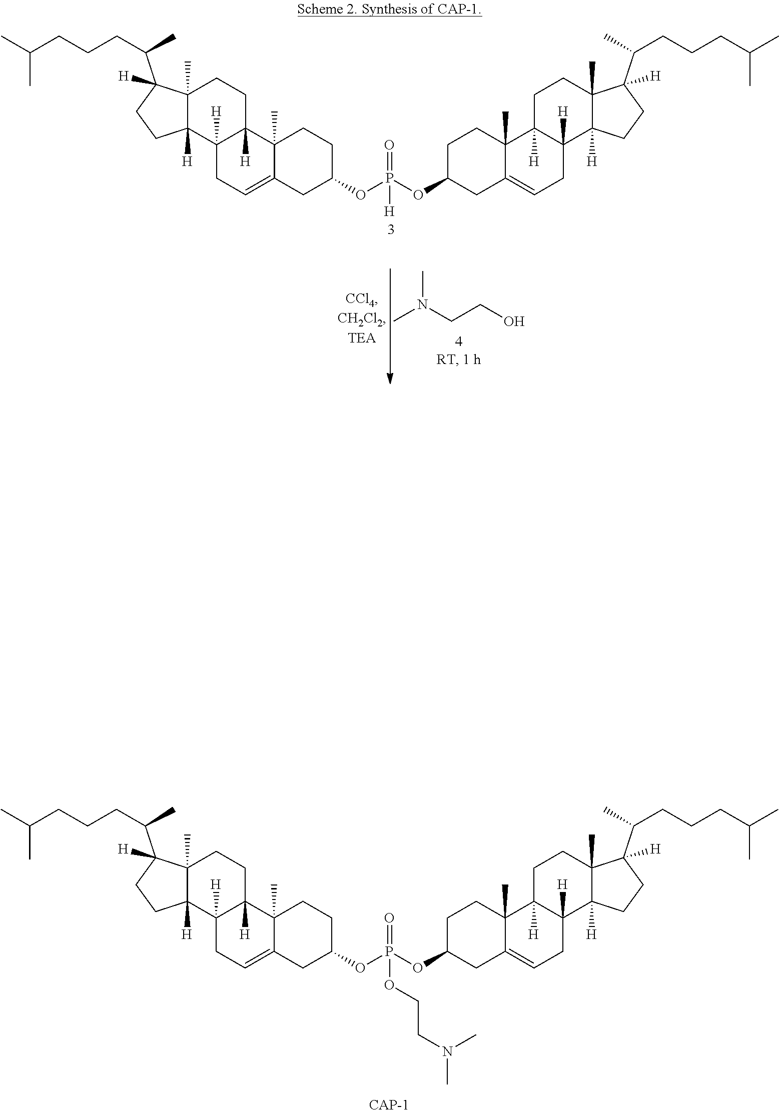

Synthesis of CAP-1. To a flame-dried flask containing 3 (286.8 mg, 0.35 mmol) and carbon tetrachloride (2.0 mL) was added dropwise a solution of trimethylamine (194.6 μL, 1.4 mmol), DMAP (4.3 mg, 0.035 mmol), and 4 (178.2 mg, 2.0 mmol) in 1.0 mL of dry DCM under vigorous stirring at RT. The reaction mixture was stirred for 1 h, diluted with 50 mL of DCM, and washed three times with 50 mL of brine. The organic phase was isolated, dried over anhydrous Na₂SO₄, filtered, and the solvent was removed in vacuo. The residue was purified via silica gel chromatography (0%-20% [mixture of 3% NH₄OH, 22% MeOH in dichloromethane] in dichloromethane). 188 mg of CAP-1 was obtained as a white powder, yield 59.3%. ³¹P NMR (121 MHz, CDCl₃) δ-2.27. ¹H NMR (300 MHz, Chloroform-d) δ 5.36 (dd, J=4.5, 2.7 Hz, 2H), 4.30-4.16 (m, 2H), 4.10 (dt, J=7.2, 6.0 Hz, 2H), 2.61 (t, J=6.0 Hz, 2H), 2.43 (d, J=6.9 Hz, 4H), 2.29 (s, 6H), 1.99 (ddt, J=13.8, 10.8, 4.2 Hz, 6H), 1.89-1.64 (m, 6H), 1.63-1.39 (m, 12H), 1.38-1.22 (m, 8H), 1.22-1.04 (m, 14H), 1.02 (d, J=3.3 Hz, 2H), 1.00 (s, 6H), 0.96 (d, J=3.3 Hz, 2H), 0.94 (s, 2H), 0.91 (d, J=6.6 Hz, 6H), 0.86 (dd, J=6.6, 1.5 Hz, 12H), 0.67 (s, 6H). MS (m/z): [M+H]⁺caled. For C₅₈H₁₀₁NO₄P, 906.7; found: 906.8.

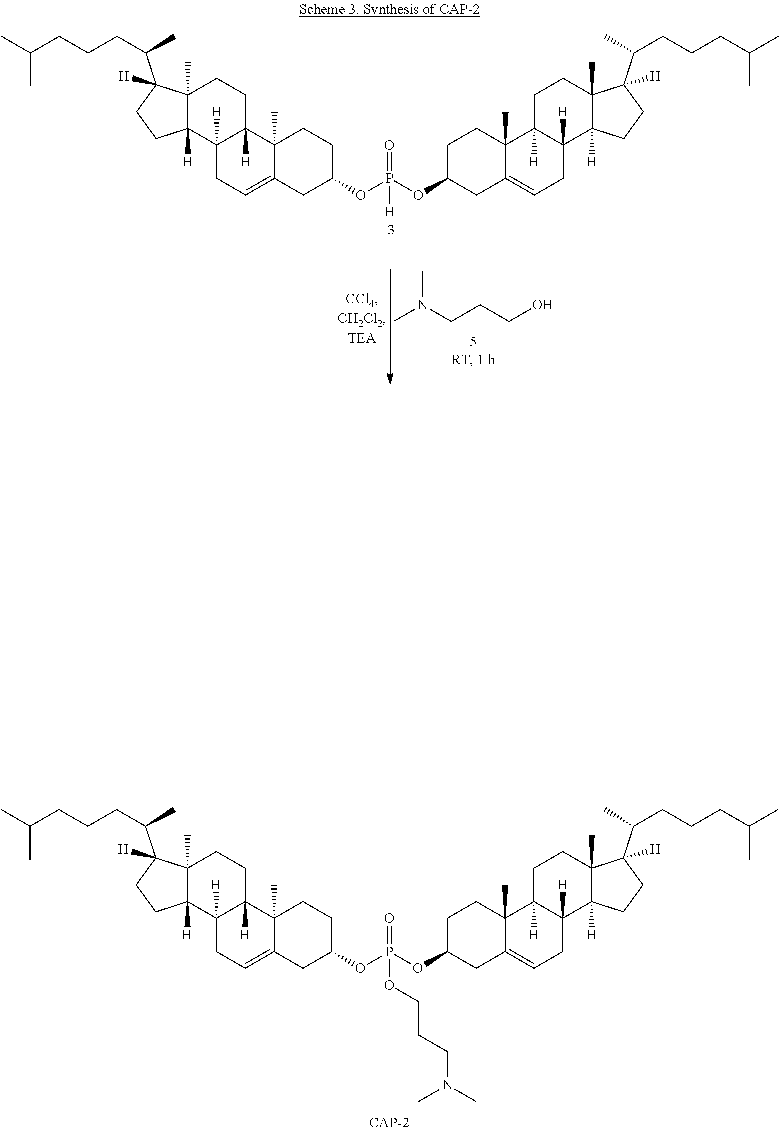

Synthesis of CAP-2. To a flame-dried flask containing 3 (286.8 mg, 0.35 mmol) and carbon tetrachloride (2.0 mL) was added dropwise a solution of trimethylamine (194.6 μL, 1.4 mmol), DMAP (4.3 mg, 0.035 mmol), and 5 (144.5 mg, 1.4 mmol) in 1.0 mL of dry DCM under vigorous stirring at RT. The reaction mixture was stirred for 1 h, diluted with 50 mL of DCM, and washed three times with 50 mL of brine. The organic phase was isolated, dried over anhydrous Na₂SO₄, filtered, and the solvent was removed in vacuo. The residue was purified via silica gel chromatography (0%-20% [mixture of 3% NH₄OH, 22% MeOH in dichloromethane] in dichloromethane). 196 mg of CAP-1 was obtained as a white powder, yield 60.8%. ³¹P NMR (121 MHZ, CDCl) δ-2.34. ¹H NMR (300 MHz, Chloroform-d) δ 5.43-5.34 (m, 2H), 4.27-4.11 (m, 2H), 4.07 (q, J=6.6 Hz, 2H), 2.41 (dd, J=12.0, 7.5 Hz, 6H), 2.24 (s, 6H), 2.06-1.91 (m, 6H), 1.83 (qd, J=9.0, 8.1, 3.0 Hz, 6H), 1.76-1.64 (m, 2H), 1.62-1.40 (m, 12H), 1.39-1.23 (m, 8H), 1.22-1.03 (m, 14H), 1.03 (s, 2H), 1.00 (s, 6H), 0.96 (d, J=3.6 Hz, 2H), 0.91 (d, J=6.6 Hz, 6H), 0.86 (dd, J=6.6, 1.4 Hz, 12H), 0.67 (s, 6H). MS (m/z): [M+H]⁺calcd. For C₅₉H₁₀₃NO₄P, 920.8; found: 920.9.

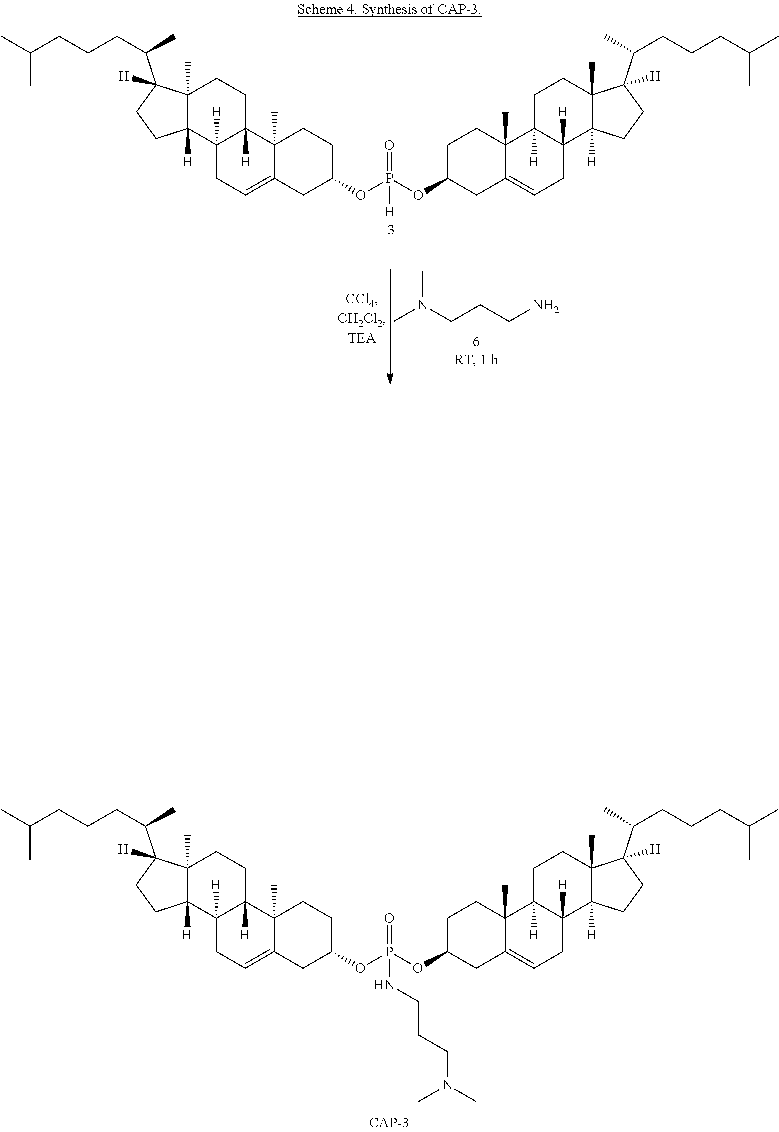

Synthesis of CAP-3. To a flame-dried flask containing 3 (286.8 mg, 0.35 mmol) and carbon tetrachloride (2.0 mL) was added dropwise a solution of trimethylamine (194.6 μL, 1.4 mmol), DMAP (4.3 mg, 0.035 mmol), and 6 (143.1 mg, 1.4 mmol) in 1.0 mL of dry DCM under vigorous stirring at RT. The reaction mixture was stirred for 1 h, diluted with 50 mL of DCM, and washed three times with 50 mL of brine. The organic phase was isolated, dried over anhydrous Na₂SO₄, filtered, and the solvent was removed in vacuo. The residue was purified via silica gel chromatography (0%-20% [mixture of 3% NH₄OH, 22% MeOH in dichloromethane] in dichloromethane). 189 mg of CAP-1 was obtained as a white powder, yield 58.7%. ³¹P NMR (121 MHz, CDCl₃) δ 7.59. ¹H NMR (300 MHz, Chloroform-d) δ 5.36 (dt, J=5.1, 2.7 Hz, 2H), 4.14 (h, J=5.4 Hz, 2H), 3.43 (dt, J=11.1, 6.6 Hz, 1H), 2.99 (dq, J=9.0, 6.6 Hz, 2H), 2.52-2.32 (m, 6H), 2.22 (s, 6H), 1.99 (ddd, J=16.5, 9.6, 6.3 Hz, 6H), 1.82 (tt, J=12.6, 4.8 Hz, 6H), 1.69-1.40 (m, 16H), 1.40-1.23 (m, 8H), 1.23-1.05 (m, 14H), 1.03 (s, 2H), 1.00 (s, 6H), 0.97 (d, J=3.0 Hz, 2H), 0.94 (s, 2H), 0.91 (d, J=6.6 Hz, 6H), 0.86 (dd, J=6.6, 1.5 Hz, 12H), 0.67 (s, 6H). MS (m/z): [M+H]⁺calcd. For C₅₉H₁₀₄N₂O₃P, 919.8; found: 919.9.

Preparation and characterization of LNPs. The mRNA and replicon were synthesized following the previously reported method (Zhang X et al. Science Advances, 2020, 6, eabc2315). The LNP formulations were prepared based on the previous reports (Vogel A B et al. Molecular Therapy 2018, 26, 446-455; Li B et al. Nano Letters 2015, 15, 8099-8107). Briefly, the ethanol phase was composed of CAP lipids or MC3 with other helper lipids dissolved in ethanol at a certain molar ratio. Aqueous phase was prepared by diluting firefly luciferase (FLuc) mRNA, Dmc1 mRNA, or mRNA replicon in a citrate buffer (pH 3). The CAP LNPs were prepared by a rapid mixing method via a pipetting technique or a microfluidic device and were then purified by dialysis. Particle size and zeta potential were quantified by NanoZS Zetasizer (Malvern). The entrapment efficiency was measured by the RiboGreen assay. The morphology of LNPs was characterized by a Cryo-EM (Thermo Scientific Glacios) device as described previously. Cell culture and in vitro luciferase assay. Hep3B cells were cultured in Eagle's minimum essential medium (Corning) with 10% fetal bovine serum (FBS). Before being treated by CAP LNPs, cells were seeded at a density of 2×10⁴ cells per well on a white 96 well flat bottom plate overnight. The dose was 50 ng Firefly luciferase mRNA per well. After 18 hours incubation, Bright-Glo luciferase (Promega) was added, and the luminescence activity was determined by Cytation 5 (Biotek).

In vivo microinjection of mouse testis and preparation of histologic sections. All animal experimental procedures were performed according to the guidelines approved by the Institutional Animal Care and Use Committee (IACUC) of the Ohio State University and complied with all relevant ethical regulations as applicable. Heterozygous mice were bred together to maintain a live colony. Dmc1″-were viable but sterile. The genotyping protocol was followed by Protocol 23094: Standard PCR Assay-Dmc1<tm 1Jcs> (JAX). The expected genotyping results were Mutant=147 bp; Heterozygote=147 bp and 233 bp; Wild type=233 bp. CAP LNPs were injected into the seminiferous tubules of mice through the previously reported microinjection method (Michaelis M et al. Journal of Visualized Experiments: JoVE, 2014, 90, e51802). For all the administrations, the mRNA concentration is 0.24 mg/ml, and the volume is 30 μl per testis. After administration, testes were harvested to prepare cryostat microtome section or meiotic chromosome spreads. For cryostat microtome section preparation, the testes were fixed by immersion in 4% paraformaldehyde in 0.1 M phosphate buffer (pH 7.4) at 4° C. for 4 h, then dehydrated in 30% sucrose, embedded in optimal cutting temperature compound (OCT compound), cut into 5-μm-thick sections using a microtome, and mounted onto glass slides. The preparation of meiotic chromosome spreads followed the previous literature (Dia F et al. Journal of Visualized Experiments: JoVE, 2017, 129, e55378). Briefly, the testes were incubated in a hypotonic solution to swell spermatocytes. Then spermatocytes are released into a sucrose solution to obtain a cell suspension, and nuclei were spread onto fixative-soaked glass slides.

Immunofluorescent staining. Firstly, the histologic sections were blocked in the donkey serum for 30 minutes. The primary antibody (dissolved in 1% BSA in 1×TBS) was then added and the slides were incubated in a humid chamber at 4° C. overnight. After incubation, the slides were washed by 1×TBS 3 times (5 minutes each time) and incubated with the secondary antibody (dissolved in 1% BSA in TBS) in the dark at room temperature. 1 h After incubation of secondary incubation, slides were washed 3 times with TBS in the dark (S minutes each time) and mounted by anti-quencher. Primary antibodies were diluted as follows: Anti-Mouse SYCP3 Antibody (1:250); Anti-Rabbit SYCP1 Antibody (1:250); Anti-Rabbit-FLAG Tag (1:100); Secondary antibodies were diluted as follows: Goat Anti-Mouse IgG H&L-Alexa Fluor® 488 (1:500); Goat Anti-Rabbit IgG H&L Alexa Fluor® 555 (1:500); PNA lectin (1:200).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A composition comprising a compound defined by Formula I, or a pharmaceutically acceptable salt thereof: 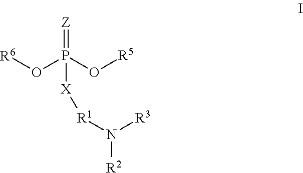
wherein
X is O, S, CH₂, or NR⁴,
Z is O or S;
R¹ is substituted or unsubstituted C₁-C₅ alkyl, substituted or unsubstituted C₁-C₅ alkenyl, or substituted or unsubstituted C₁-C₈ alkynyl;
R² and R³ are independently hydrogen or substituted or unsubstituted C₁-C₅ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety;
R⁴, when present, is hydrogen or substituted or unsubstituted C₁-C₈ alkyl; and
R⁵ and R⁶ are independently a sterol.

2. The composition of claim 1, wherein R⁵ and R⁶ are each independently selected from the group consisting of cholesterol, β-sitosterol, fucosterol, campesterol, ergosterol, stigmastanol, brassicasterol, and derivatives thereof.

3. The composition of claim 1, wherein R⁵ and R⁶ are the same.

4. (canceled)

5. The composition of claim 1, wherein the compound is defined by Formula II: 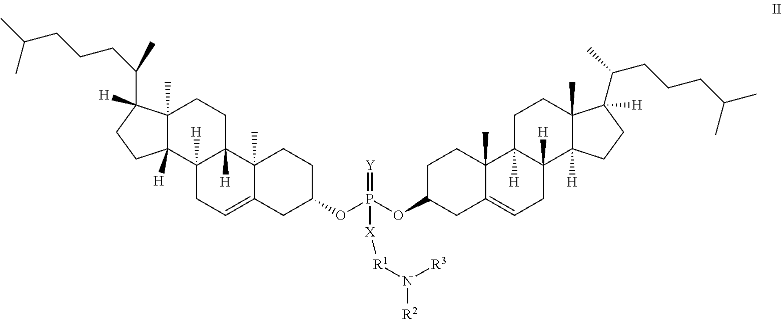
or a pharmaceutically acceptable salt thereof.

6. (canceled)

7. The composition of claim 1, wherein the compound is defined by Formula III, or a pharmaceutically acceptable salt thereof: 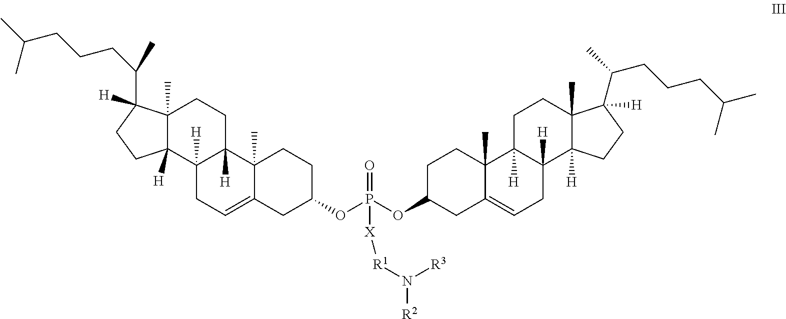
wherein
X is O, S, CH₂, or NR⁴;
R¹ is substituted or unsubstituted C₁-C₅ alkyl, substituted or unsubstituted C₁-C₅ alkenyl, or substituted or unsubstituted C₁-C₅ alkynyl;
R² and R³ are independently hydrogen or substituted or unsubstituted C₁-C₈ alkyl, substituted or unsubstituted C₁-C₅ cycloalkyl, or wherein, as valence permits, R² and R³ together with the atoms to which they are attached, for a 3-10 membered substituted or unsubstituted heterocyclic moiety; and
R⁴, when present, is hydrogen or substituted or unsubstituted C₁-C₈ alkyl.

8. The composition of claim 1, wherein R¹ is substituted or unsubstituted C₁-C₈ alkyl.

9. (canceled)

10. (canceled)

11. The composition of claim 1, wherein the compound is defined by Formula IV, or a pharmaceutically acceptable salt thereof: 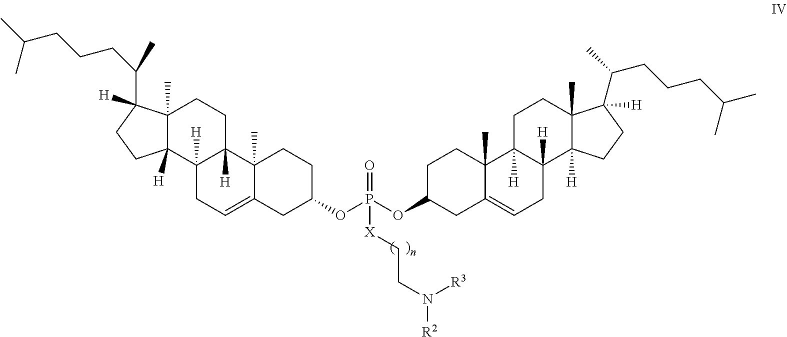
wherein n is an integer from 1 to 5.

12. The composition of claim 1, wherein R² and R³ are each independently substituted or unsubstituted C₁-C₅ alkyl.

13. (canceled)

14. (canceled)

15. The composition of claim 1, wherein R² and R³ are the same.

16. (canceled)

17. The composition of claim 1, wherein the compound is defined by Formula V, or a pharmaceutically acceptable salt thereof: 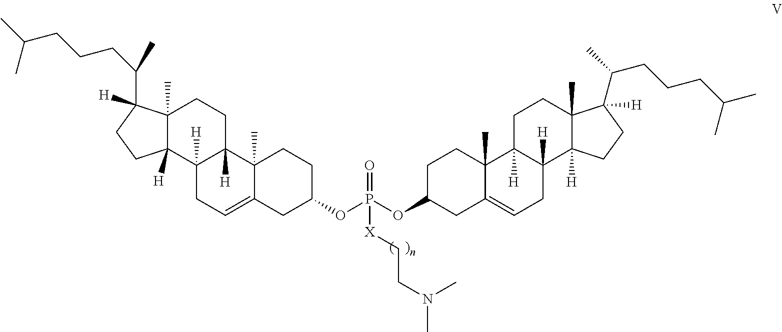
wherein n is an integer from 1 to 5.

18.-25. (canceled)

26. The composition of claim 1, wherein the compound is selected from the group consisting of: 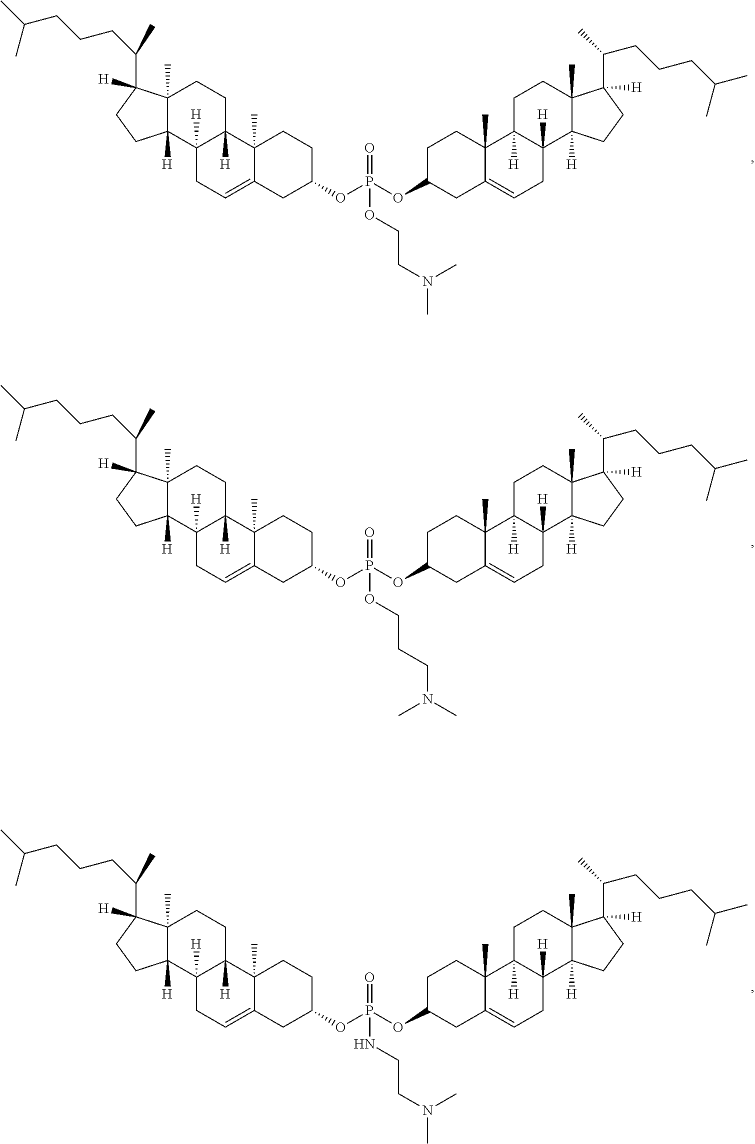 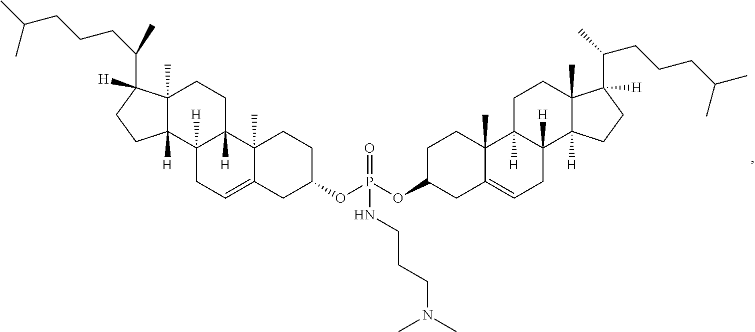
pharmaceutically acceptable salts thereof, and combinations thereof.

27. (canceled)

28. (canceled)

29. (canceled)

30. A lipid particle comprising the composition of claim 1.

31.-38. (canceled)

39. A pharmaceutical composition comprising a therapeutic agent encapsulated within the lipid particle of claim 30.

40. (canceled)

41. (canceled)

42. (canceled)

43. The pharmaceutical composition of claim 39, wherein the therapeutic agent comprises a nucleic acid.

44. The pharmaceutical composition of claim 43, wherein the nucleic acid is mRNA, saRNA, or a combination thereof.

45. The pharmaceutical composition of claim 44, wherein the nucleic acid encodes a protein.

46. The pharmaceutical composition of claim 44, wherein the nucleic acid encodes DNA Meiotic Recombinase 1 (Dmc1) protein.

47. (canceled)

48. A method of treating a disease or disorder in a subject in need thereof, the method comprising administering to the subject a therapeutically effective amount of the pharmaceutical composition of claim 39.

49. The method of claim 48, wherein the subject is a human male and the disease or disorder comprises male infertility.

50. The method of claim 49, wherein the method comprises delivering the therapeutically effective amount of the pharmaceutical composition to the testis or spermatocytes.

51. (canceled)
